(19) United States
(12) Patent Application Publication
(10) Pub. No.: US 2025/0108095 A1
(43) Pub. Date: Apr. 3, 2025

(54) DEGRADATION OF TOXINS IN VIVO

(71) Applicant: Aperiam Bio, Inc., Brooklyn, NY (US)

(72) Inventors: Austin Woodrow Cole, Brooklyn, NY (US); Guillaume Cottarel, Durham, NC (US)

(73) Assignee: Aperiam Bio, Inc., Brooklyn, NY (US)

(21) Appl. No.: 18/899,962

(22) Filed: Sep. 27, 2024

Related U.S. Application Data
(60) Provisional application No. 63/541,229, filed on Sep. 28, 2023.

(51) Int. Cl. A61K 38/44; A61K 38/45; A61K 38/50; A61K 38/51; A61K 38/53

(52) U.S. Cl. CPC A61K 38/44; A61K 38/45; A61K 38/50; A61K 38/51; A61K 38/53; C12Y 107/03003; C12Y 208/01001; C12Y 305/01001; C12Y 305/03001; C12Y 403/01024; C12Y 603/01002

(57) ABSTRACT

The present disclosure generally relates to technologies for the degradation a systemic toxin in vivo by enhancing the metabolic functionality of the lungs. Such technologies comprise introducing, to pulmonary tissue of a subject, a composition comprising at least one enzyme known to enzymatically break down at least one toxin present systemically in the subject. The provided technologies enable the lung to behave as a tunable metabolic organ, facilitating the removal of toxins from the systemic circulation of a subject, such as a subject having a condition resulting in the toxic accumulation or an endogenous metabolite, and a subject who has consumed a toxic substance.

### CROSS-REFERENCE TO RELATED APPLICATIONS

This application claims the benefit under 35 U.S.C. § 119 (e) of U.S. Provisional Patent Application No. 63/541,229, filed Sep. 28, 2023, the entire contents of which is incorporated herein by reference in its entirety.

### BACKGROUND

A multitude of human diseases and conditions are caused by the accumulation of toxic substances in the body. Some genetic disorders in which a human expresses a dysfunctional or non-functional enzyme that would otherwise act on a natural metabolite can result in the toxic accumulation of that metabolite. Other diseases and conditions are caused by purposeful or accidental poisoning events. Common to all of these illnesses is the excess buildup of a chemical substance in the body of the subject. In many cases, the toxic accumulation of such molecules occurs in the systemic circulation of the subject.

Genetic conditions can result in the toxic systemic accumulation of certain macromolecules, the degradation or metabolism of which are important for normal biological function and health. For example, Phenylketonuria (PKU) is an inherited metabolic disorder caused by an inactive or deficient phenylalanine hydroxylase (PAH). PAH is required for the breakdown of phenylalanine (Phe), an amino acid found in all protein-containing foods. When PAH is deficient or inactive, Phe accumulates to abnormally high levels in the blood. Clinical manifestations of sustained high Phe levels include a variety of serious neurological and neuropsychological complications. Dietary changes are often the first line of disease management, including a low-protein diet and specially formulated Phe-free medical foods. However, changes in diet are often insufficient to reduce the negative health impacts of sustained systemic elevations in Phe levels.

The systemic accumulation of toxic molecules can also result from the introduction of exogenous substances into the circulation of a subject. For example, parathion is an organophosphate insecticide with an oral human LD50 of 8 mg/kg, and is readily absorbed by the skin, mucous membranes, and by oral ingestion. Parathion directly and stoichiometrically inactivates acetylcholinesterase in humans by covalently bonding with the active site. Without intervention, parathion poisoning can be fatal. An estimated 3 million or more people worldwide are exposed to organophosphates each year, accounting for about 300,000 deaths. Thus, organophosphate exposure, as well as exposure to other classes of toxins, remain serious public health concerns.

US20220047682A1 describes an amino acid sequence encoding ADH/KRED bound to at least one long-acting molecule or complexing molecule. The long-acting alcohol dehydrogenase disclosed is described as having extended circulatory half-lives, higher area under the curve value, lower clearance value, lower elimination rate and higher t½ measure within the blood and serum compared to wild-type ADH.

### SUMMARY OF THE INVENTION

In one aspect, the present disclosure provides a method of alleviating one or more negative effects of a toxin in vivo comprising: introducing at least one preselected enzyme to pulmonary tissue of a subject, said at least one preselected enzyme being known to enzymatically breakdown at least one toxin that is exerting one or more negative effects against said subject; and allowing the enzymatic breakdown of said at least one toxin to take place in vivo, alleviating said one or more negative effects. In some embodiments, said subject is afflicted with one or more genetic disorders. In some embodiments, said one or more genetic disorders prevent a breakdown of said at least one toxin. In some embodiments, said one or more genetic disorders is selected from the group consisting of phenylketonuria, gout, cystinuria, ornithine transcarbamylase deficiency (OTCD), galactosemia, maple syrup urine disease, tumor suppression disorder, cocaine use disorder, urea cycle disorder, tobacco use disorder, Pompe's disease, sucrase isomaltase deficiency, arginase deficiency, hyperargininemia, and combinations thereof. In some embodiments, said subject suffers from a condition that results in an accumulation of said at least one toxin. In some embodiments, said subject has been exposed to said at least one toxin. In some embodiments, said at least one toxin is selected from the group consisting of phenylalanine, uric acid, cystine, arginine, lysine, ornithine, leucine, isoleucine, valine, an amino acid, galactose, kynurenine, cocaine, ammonia, nicotine, cyanide, organophosphates, and combinations thereof. In some embodiments, said at least one preselected enzyme is selected from the group consisting of phenylalanine ammonia lyase (PAL), phenylalanine hydroxylase (PAH), galactose degrading enzyme, galactose-1-phosphate uridylyltransferase (GALT), 2-oxoisovalerate dehydrogenase subunit alpha, mitochondrial (BCKDHA), 2-oxoisovalerate dehydrogenase subunit beta, mitochondrial (BCKDHB), BDT complex enzymes, kynureninase, cocaine esterase, arginase, glutamine synthase, arginosuccinate lyase, arginosuccinate synthase, carbamyl phosphate synthetase I, N-acetylglutamate synthetase, ornithine transcarbamylase, ornithine translocase, nicotine oxidoreductase, uricase, acid alpha-glucosidase (GAA), thiosulfate sulfurtransferase (rhodanese), collagenase, asparaginase, anti-inhibitor coagulant complex, tissue-type plasminogen activator (tPa), Alteplase, pegademase bovine, alglucerase, imiglucerase, Factor IX, dnase, pancrelipase (amylase; lipase; protease), sacrosidase, truncated (non-glycosylated) t-PA (357 of 527aa), coagulation Factor VIIa, tissue plasminogen activator, antihemophilic factor (AHF), laronidase, agalsidase beta, hyaluronidase, galsulfase, idursulfase, alglucosidase alfa, thrombin, velaglucerase alfa, pegloticase, asparaginase, taliglucerase alfa, plasmin, carboxypeptidase g2, glucarpidase, coagulation Factor XIII A, closulfase alfa, coagulation Factor X, asfotase alfa, sebelipase alfa, cerliponase alfta, vestronidase alfa-vjbk, pegvaliase-pqpz, and combinations thereof. In some embodiments, said at least one toxin is an amino acid (phenylalanine or Phe) or an acid (uric acid). In some embodiments, said pulmonary tissue exposes said at least one toxin by an action of the subject's circulatory system. In some embodiments, said introducing step comprises contacting pulmonary tissue with either a polypeptide having an amino acid sequence corresponding to said at least one preselected enzyme, a polynucleotide encoding said at least one polypeptide, or both. In some embodiments, said introducing step comprises transducing a plurality of cells present in pulmonary tissue to express said at least one preselected enzyme. In some embodiments, the plurality of cells is transduced with DNA or a construct thereof. In some embodiments, the plurality of cells is transduced with mRNA or a construct thereof. In some embodiments, allowing the enzymatic breakdown of said at least one toxin comprises allowing the degradation of said at least one toxin that has diffused or migrated from the subject's circulatory system into the subject's lung and/or lung mucous.

In one aspect, the present disclosure provides a method of alleviating one or more negative effects of at least one toxin in vivo comprising introducing at least one preselected enzyme to pulmonary tissue of a subject, said at least one preselected enzyme known to enzymatically breakdown at least one toxin that is exerting one or more negative effects against said subject. In some embodiments, said at least one preselected enzyme is selected from the group consisting of phenylalanine ammonia lyase (PAL), phenylalanine hydroxylase (PAH), galactose degrading enzyme, galactose-1-phosphate uridylyltransferase (GALT), 2-oxoisovalerate dehydrogenase subunit alpha, mitochondrial (BCKDHA), 2-oxoisovalerate dehydrogenase subunit beta, mitochondrial (BCKDHB), BDT complex enzymes, kynureninase, cocaine esterase, arginase, glutamine synthase, arginosuccinate lyase, arginosuccinate synthase, carbamyl phosphate synthetase I, N-acetylglutamate synthetase, ornithine transcarbamylase, ornithine translocase, nicotine oxidoreductase, uricase, acid alpha-glucosidase (GAA), thiosulfate sulfurtransferase (rhodanese), collagenase, asparaginase, anti-inhibitor coagulant complex, tissue-type plasminogen activator (tPa), Alteplase, pegademase bovine, alglucerase, imiglucerase, Factor IX, dnase, pancrelipase (amylase; lipase; protease), sacrosidase, truncated (non-glycosylated) t-PA (357 of 527aa), coagulation Factor VIIa, tissue plasminogen activator, antihemophilic factor (AHF), laronidase, agalsidase beta, hyaluronidase, galsulfase, idursulfase, alglucosidase alfa, thrombin, velaglucerase alfa, pegloticase, asparaginase, taliglucerase alfa, plasmin, carboxypeptidase g2, glucarpidase, coagulation Factor XIII A, closulfase alfa, coagulation Factor X, asfotase alfa, sebelipase alfa, cerliponase alfta, vestronidase alfa-vjbk, pegvaliase-pqpz, and combinations thereof. In one embodiment of the disclosure, the at least one preselected enzyme is not ADH/KRED bound to at least one long-acting molecule or complexing molecule. In some embodiments, the method further comprises allowing the degradation of said at least one toxin that has diffused or migrated from the subject's circulatory system into the subject's lung and/or lung mucous to take place in vivo, alleviating said one or more negative effects.

In one aspect, the present disclosure provides a method of transforming a lung of a subject to include at least one enzymatic breakdown function comprising introducing to a lung of a subject at least one preselected enzyme known to facilitate an enzymatic breakdown of at least one toxin that, if present, is present systemically in the subject. In some embodiments, the at least one toxin is circulating within the subject, including the subject's lung. In some embodiments, the method further comprises allowing said at least one preselected enzyme to come into contact with said at least one toxin, facilitating its enzymatic breakdown in vivo. In some embodiments, the enzymatic breakdown is facilitated in the subject's lung and/or lung mucous.

In one aspect, the present disclosure provides a transformed lung capable of enzymatically breaking down at least one toxin that is systemically present in a subject, including the subject's lung, comprising pulmonary tissue transformed to harbor at least one preselected enzyme known to enzymatically breakdown the at least one toxin. In some embodiments, said transformed pulmonary tissue harbors at least one preselected enzyme via an introduction of either a polypeptide having an amino acid sequence corresponding to said preselected enzyme, a polynucleotide encoding said polypeptide, or both.

In one aspect, the present disclosure provides a transformed pulmonary cell comprising an alveolar cell harboring at least one preselected enzyme known to enzymatically breakdown at least one toxin. In some embodiments, said transformed alveolar cells harbors at least one preselected enzyme via an introduction of either a polypeptide having an amino acid sequence corresponding to said preselected enzyme, a polynucleotide encoding said polypeptide, or both.

In one aspect, the present disclosure provides a population of pulmonary cells comprising a plurality of alveolar cells harboring at least one preselected enzyme known to enzymatically breakdown at least one toxin.

In one aspect, the present disclosure provides a method of alleviating one or more negative effects of leukemia in a subject, comprising: introducing asparaginase to pulmonary tissue of a subject diagnosed with leukemia, said asparaginase being known to enzymatically breakdown asparagine; and allowing the enzymatic breakdown of asparagine to take place in vivo, alleviating said one or more negative effects. In some embodiments, said leukemia comprises acute lymphoblastic leukemia (ALL). In some embodiments, said asparaginase comprises L-asparaginase.

In one aspect, the present disclosure provides a method of alleviating one or more negative effects of excess oxalate in a subject, comprising: introducing oxalate decarboxylase to pulmonary tissue of a subject diagnosed with oxalosis, primary hyperoxaluria, or secondary hyperoxaluria, said oxalate decarboxylase known to enzymatically breakdown oxalate; and allowing the enzymatic breakdown of said oxalate. In some embodiments, said oxalate comprises endogenously produced or dietary oxalate.

In one aspect, the present disclosure provides a method of alleviating one or more negative effects of phenylketonuria in a subject, comprising: introducing phenylalanine ammonia lyase (PAL) to pulmonary tissue of a subject diagnosed with phenylketonuria, said PAL known to enzymatically breakdown phenylalanine; and allowing the enzymatic breakdown of said phenylalanine. In some embodiments, the subject comprises a mutation in a phenylalanine hydroxylase (PAH) gene.

In one aspect, the present disclosure provides a method of alleviating one or more negative effects of hyperuricemia in a subject, comprising: introducing uricase to pulmonary tissue of a subject diagnosed with hyperuricemia, said uricase known to enzymatically breakdown uric acid; and allowing the enzymatic breakdown of said uric acid. In some embodiments, the subject has overproduction of uric acid. In some embodiments, the subject has underexcretion of uric acid. In some embodiments, the subject is diagnosed with or suspected of having gout.

In one aspect, the present disclosure provides a method of alleviating one or more negative effects of cyanide poisoning in a subject, comprising: introducing thiosulfate sulfurtransferase (rhodanese) to pulmonary tissue of a subject diagnosed with cyanide poisoning, said rhodanese known to enzymatically breakdown cyanide; and allowing the enzymatic breakdown of said cyanide. In some embodiments, the subject has ingested a cyanide salt, has consumed liquid prussic acid, has absorbed prussic acid through the skin, has been intravenously administered nitroprusside, or has inhaled hydrogen cyanide gas. In some embodiments, introducing further comprises introducing sodium thiosulfate to pulmonary tissue of the subject.

In one aspect, the present disclosure provides a method of alleviating one or more negative effects of hyperammonemia in a subject, comprising: introducing glutamine synthase to pulmonary tissue of a subject diagnosed with hyperammonemia, said glutamine synthase known to enzymatically remove ammonia; and allowing the enzymatic removal of said ammonia. In some embodiments, the subject has overproduction of ammonia. In some embodiments, the subject has underexcretion of ammonia. In some embodiments, the subject is diagnosed with or suspected of having Ornithine transcarbamylase deficiency (OTCD).

In one aspect, the present disclosure provides a method of alleviating one or more negative effects of hyperargininemia in a subject, comprising: introducing arginase to pulmonary tissue of a subject diagnosed with hyperargininemia, said arginase known to enzymatically breakdown arginine; and allowing the enzymatic breakdown of said arginine. In some embodiments, the subject has overproduction of arginine. In some embodiments, the subject is diagnosed with or suspected of having arginase deficiency.

In some embodiments, said at least one preselected enzyme, a variant thereof, or a combination thereof resides substantially in the subject's pulmonary tissue.

In some embodiments, said at least one preselected enzyme, a variant thereof, or a combination thereof is introduced under conditions that inhibit or do not support systemic delivery of said enzyme, a variant thereof, or a combination thereof to said subject.

In some embodiments, said at least one preselected enzyme, a variant thereof, or a combination thereof is introduced in a manner that does not include pulmonary transmucosal delivery of said at least one preselected enzyme, a variant thereof, or a combination thereof to said subject.

In some embodiments, said at least one preselected enzyme, a variant thereof, or a combination thereof is introduced to the at least a portion of the lung of said subject in a manner that minimizes a systemic introduction into said subject.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF THE DRAWINGS

FIG. 1 is a graph illustrating the effect of the route of administration of phenylalanine ammonia lyase (PAL) on circulating phenylalanine (Phe) levels over time in a chemically-induced rat model of phenylketonuria (PKU), measured in units of nmol/mL of serum.

FIG. 2A is a graph illustrating the effect of the route of administration of phenylalanine ammonia lyase (PAL) on circulating phenylalanine (Phe) levels in a chemically-induced mouse model of PKU, measured in units of μg/mL of serum.

FIG. 2B is a graph illustrating the change in circulating Phe levels as a function of administration route of PAL administration in mice, measured in units of μg/mL of serum. The y-axis displays the difference between the Phe measurement (reported as μg/mL) obtained after PAL administration and that obtained before PAL administration.

FIG. 3 is a graph illustrating the effect of the route of administration of phenylalanine ammonia lyase (PAL) on circulating phenylalanine (Phe) levels in a genetic mouse model of PKU, measured in μM. Grey boxes signify IT administration of PAL; blue boxes signify subcutaneous (SQ) administration of PAL.

FIG. 4 is a graph illustrating serum uric acid concentration (μM) in serum samples obtained from mice administered a uricase enzyme as a function of time following administration. The measurement at time 0 was obtained immediately prior to uricase administration. IT=Intratracheal administration; IV=Intravenous administration; black circles=no delivered uricase; red circles=IV-administered uricase; blue circles=IT-delivered uricase.

FIG. 5A is a graph illustrating serum uric acid concentration (μM) in serum samples obtained from mice administered a uricase enzyme as a function of time following administration. Healthy female mice were provided with either 2 mg/mouse of A. flavus uricase, delivered IV (green; n=5) or IT (blue; n=5), or with IT-delivered saline (black, n=2). Time course data of serum uric acid (UA) post-dose. Both IV and IT uricase significantly decreased UA. Vehicle administration did not change UA levels throughout the study. Statistics: data shown as mean+SD; multiple t-test comparing Control to Treatment.

FIG. 5B is a graph illustrating the Area Under the Curve (AUC) of time course data described in FIG. 5A. Statistics: data shown as mean+SD; One-way ANOVA comparing Control to Treatment (*). Directional black arrows indicate percent decrease compared to Control. Unpaired t-test comparing Treatment groups (\hat{ }).

FIG. 6A is a graph illustrating serum uric acid concentration (μM) in serum samples obtained from Cynomolgus non-human primates (NHP) administered a variant uricase enzyme as a function of time following administration. At Cₘₐₓ, UA serum concentration was 74 μM±24.5 SD (UA Control), 59 μM±12 SD (Low-dose uricase+UA), and 46 μM±20 SD (High-dose uricase+UA).

FIG. 6B is a graph illustrating the Area Under the Curve (AUC) of time course data described in FIG. 6A. Serum uric acid was decreased 36.6% (Low-dose uricase) and 56.8% (High-dose uricase, p<0.01) compared to UA Control, as determined by area under the curve analysis of time course data (60-515 minutes). Data are shown as mean+SD, One-way ANOVA vs Uric Acid Control. **p<0.01, ***p<0.001.

FIG. 7A is a graph illustrating serum uric acid concentration (μM) in serum samples obtained from mice administered a wildtype uricase or a variant uricase enzyme as a function of time following administration. Healthy female mice were provided with either 2 mg/mouse of A. flavus uricase or engineered variant uricase, or vehicle (black, n=3). Time course data of serum uric acid (UA) post-dose is shown. Statistics: data shown as mean+SD; multiple t-test comparing Control to Treatment.

FIG. 7B is a graph illustrating the Area Under the Curve (AUC) of time course data described in FIG. 7A. Serum uric acid was decreased 29.8% (WT uricase) and 34.4% (variant uricase, p<0.01) compared to UA Control, as determined by area under the curve analysis of time course data (0-240 minutes). Statistics: data shown as mean+SD; One-way ANOVA comparing Control to Treatment groups (*). Directional black arrows indicate percent decrease compared to Control. Unpaired t-test comparing Treatment groups (\hat{ }).

FIG. 8A is a graph illustrating plasma ammonia (μM) levels in samples obtained from mice administered saline followed by NH4Cl (triangles), a wildtype glutamine synthetase (GS) enzyme followed by NH4Cl (crosses), or a wildtype glutamine synthetase (GS) enzyme plus glutamate followed by NH4Cl (circles), as a function of time following administration.

FIG. 8B is a graph illustrating plasma glutamine (μM) levels in samples obtained from mice administered saline followed by NH4Cl (triangles), a wildtype glutamine synthetase (GS) enzyme followed by NH4Cl (crosses), or a wildtype glutamine synthetase (GS) enzyme plus glutamate followed by NH4Cl (circles), as a function of time following administration.

FIG. 8C is a graph illustrating plasma glutamate (μM) levels in samples obtained from mice administered saline followed by NH4Cl (triangles), a wildtype glutamine synthetase (GS) enzyme followed by NH4Cl (crosses), or a wildtype glutamine synthetase (GS) enzyme plus glutamate followed by NH4Cl (circles), as a function of time following administration.

FIG. 9 is a graph illustrating plasma ammonia (μM) levels in samples obtained from WT mice administered saline followed by NH4Cl (crosses), OTCD model mice administered saline followed by NH4Cl (circles), and OTCD model mice administered a wildtype glutamine synthetase (GS) enzyme followed by NH4Cl (triangles), as a function of time following administration.

FIG. 10A is a graph illustrating plasma L-arginine (μM) levels in samples obtained from mice administered saline (squares) or an arginase enzyme (circles) as a function of time following administration.

FIG. 10B is a graph illustrating plasma urea (mM) levels in samples obtained from mice administered saline (squares) or an arginase enzyme (circles) as a function of time following administration.

FIG. 11A is a graph illustrating plasma asparagine (μM) levels in samples obtained from mice administered saline (squares) or an asparaginase enzyme (circles) as a function of time following administration.

FIG. 11B is a graph illustrating plasma aspartate (μM) levels in samples obtained from mice administered saline (squares) or an asparaginase enzyme (circles) as a function of time following administration.

FIG. 11C is a graph illustrating plasma ammonia (μM) levels in samples obtained from mice administered saline (squares) or an asparaginase enzyme (circles) as a function of time following administration.

### DETAILED DESCRIPTION

Systemic toxicity is a condition caused as a result of build-up and/or absorption and distribution of a substance that affects the whole body rather than a specific (local) area. For example, systemic toxicity can result from one or more genetic disorders in which an enzyme responsible for the breakdown or clearance of an endogenous molecule or metabolite is hindered or abrogated, resulting in the accumulation of a substance in the systemic circulation that can impair certain physiological processes. Systemic toxicity can also result from the consumption of and subsequent absorption of a toxic substance into the systemic circulation of a subject. Thus, toxicity in the systemic circulation can be viewed as a fundamental contributor to the etiology of a diverse set of diseases and conditions. In one embodiment of the disclosure, a toxic substance excludes an alcohol, e.g., ethanol.

In view the fact that systemic toxicity plays a fundamental role in the pathophysiology of a wide array of diseases and conditions, a need remains for novel compositions, methods, and technologies for the in vivo degradation toxic substances present in the systemic circulation of a subject.

The present disclosure generally relates to, technologies for degrading a systemic toxin of a subject in vivo. Such technologies comprise introducing, to pulmonary tissue of a subject, a composition comprising at least one enzyme known to enzymatically break down at least one toxin present systemically in the subject (e.g., to reduce the level of the toxin in the subject, e.g., the level of a toxic metabolite endogenous to the subject, or a toxin that has been exogenously consumed by the subject). It should be understood that an introduction of at least one preselected enzyme, includes an introduction of at least one preselected variant thereof or a combination of at least one preselected enzyme and at least one preselected variant thereof.

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. Although not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety. In case of conflict, the present specification, including definitions, will control. Other aspects are set forth within the claims that follow.

The practice of the present technology will employ, unless otherwise indicated, conventional techniques of tissue culture, immunology, molecular biology, microbiology, chemical engineering, and cell biology, which are within the skill of the art.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations that can be varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”.

### Definitions

As used in the description of the invention and the appended claims, the singular forms “a,” “an” and “the” are intended to include the plural forms as well, unless the context clearly indicates otherwise.

The terms “substantially” and “about” are used herein to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. The terms or “acceptable,” “effective,” or “sufficient” when used to describe the selection of any components, ranges, dose forms, etc. disclosed herein intend that said component, range, dose form, etc. is suitable for the disclosed purpose.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “optional” or “optionally” means that the subsequently described event or circumstance can or cannot occur, and that the description includes instances where the event or circumstance occurs and instances where it does not.

As used herein, “administering” or the “administration,” “introducing” or the “introduction” of an agent (i.e., a therapeutic agent, including a prescription or a non-prescription drug), drug product (including a composition (i.e., a formulation or medicament)), a dietary supplement, a food, or a cosmetic product to a subject includes any route of introducing or delivering to a subject a product to perform its intended function. Administration or introduction to pulmonary tissue of a subject may be carried out by any suitable route but under conditions that do not support a significant or substantial permeation of the agent beyond pulmonary tissue (i.e., minimization of systemic introduction) . . . . Administration or introduction can be carried out by inhalation. However, to an extent significant or substantial systemic introduction is to be avoided, inhalation of an atomized solution of an agent is preferably avoided. Avoiding or minimization of systemic introduction means that a majority of the agent introduced continues to reside in pulmonary tissue. In certain embodiments of this disclosure, less than about 50%, less than about 40%, less than about 30%, less than about 20%, or less than about 10% of the agent introduced permeates mucosal tissue (i.e., introduction is not transmucosal) into a subject's blood or serum. Administration or introduction can be carried out intratracheally, the agent(s) introduced eventually finding their way into pulmonary tissue of a subject. Alternatively, administration or introduction may be carried out topically, intranasally, but preferably not intraperitoneally, intradermally, ophthalmically, intrathecally, intracerebroventricularly, iontophoretically, transmucosally, intravitreally, or intramuscularly. Administration includes self-administration, the administration by another or administration by use of a device (e.g., a vaper, a dry powder inhaler, or vapor pump, but preferably not an infusion pump). In one embodiment of the disclosure, introduction excludes pulmonary transmucosal delivery of an agent, which leads to systemic delivery in the subject. In some embodiments, an introduction of an ADH/KRED bound to at least one long-acting molecule or complexing molecule is excluded. In still other embodiments, administration excludes an inhalation of an atomized solution of an ADH/KRED bound to at least one long-acting molecule or complexing molecule.

As used herein, to “ameliorate” or “ameliorating” a disease, disorder or condition refers to results that, in a statistical sample or specific subject, make the occurrence of the disease, disorder or condition (or a sign, symptom or condition thereof) better or more tolerable in a sample or subject administered a therapeutic agent relative to a control sample or subject.

As used herein, the term “amino acid” includes both a naturally occurring amino acid and a non-natural amino acid. The term “amino acid,” unless otherwise indicated, includes both isolated amino acid molecules (i.e., molecules that include both, an amino-attached hydrogen and a carbonyl carbon-attached hydroxyl) and residues of amino acids (i.e., molecules in which either one or both an amino-attached hydrogen or a carbonyl carbon-attached hydroxyl are removed). The amino group can be alpha-amino group, beta-amino group, etc. For example, the term “amino acid alanine” can refer either to an isolated alanine H-Ala-OH or to any one of the alanine residues H-Ala-, -Ala-OH, or -Ala-. Unless otherwise indicated, all amino acids found in the agents described herein can be either in D or L configuration. An amino acid that is in D configuration may be written such that “D” precedes the amino acid abbreviation. For example, “D-Arg” represents arginine in the D configuration. According to convention, if there is no “D” or “L” that precedes the amino acid, the amino acid is assumed to be of the “L” configuration. Notably, many amino acid residues are commercially available in both D- and L-form.

The term “amino acid” includes salts thereof, including pharmaceutically acceptable salts. Any amino acid can be protected or unprotected. Protecting groups can be attached to an amino group (for example alpha-amino group), the backbone carboxyl group, or any functionality of the side chain. As an example, phenylalanine protected by a benzyloxycarbonyl group (Z) on the alpha-amino group would be represented as Z-Phe-OH. Amino acid protecting groups are well known in the art. A comprehensive review of amino acid protecting groups can be found in: Isidro-Llobet et al., Chem. Rev. (2009) 109:2455-2504.

With the exception of the N-terminal amino acid, all abbreviations of amino acids (for example, Phe) in this disclosure stand for the structure of —NH—C(R)(R′)—CO—, wherein R and R′ each is, independently, hydrogen or the side chain of an amino acid (e.g., R=benzyl and R′═H for Phe). Accordingly, phenylalanine is H-Phe-OH. The designation “OH” for these amino acids, or for peptides (e.g., Lys-Val-Leu-OH) indicates that the C-terminus is the free acid. The designation “NH₂” in, for example, H-Phe-D-Arg-Phe-Lys-NH₂ indicates that the C-terminus of the protected peptide fragment is amidated. In each case, an “H” preceding an amino acid or peptide indicates that the amine of the amino acid or peptide N-terminus is unmodified (i.e. is —NH₂). Further, certain R and R′, separately, or in combination as a ring structure, can include functional groups that may require protection during the liquid phase or solid phase synthesis.

As used herein the terms “carrier,” “pharmaceutically acceptable carrier,” “physiologically acceptable carrier,” or “cosmetically acceptable carrier” refer to a diluent, adjuvant, excipient, or vehicle with which a drug product/composition (including a formulation or medicament) is administered or formulated for administration. Non-limiting examples of such pharmaceutically acceptable carriers include liquids, such as water, saline, oils and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the term “effective amount” refers to a quantity of a composition/drug product sufficient to achieve a desired therapeutic and/or prophylactic effect, e.g., an amount that treats, prevents, inhibits, ameliorates, or delays the onset of the disease, disorder or condition, or the physiological signs, symptoms or conditions of the disease or disorder. In the context of therapeutic or prophylactic applications, in some embodiments, the amount of a composition/drug product administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors. The compositions/drug products can also be administered in combination with one or more additional therapeutic agents (a so called “co-administration” where, for example, the additional or other therapeutic agent(s) could be administered simultaneously, sequentially or by separate administration).

As used herein, the term “pharmaceutical composition” refers to the combination of a therapeutic agent with a carrier, inert or active, making the composition especially suitable for therapeutic use in vivo. By the same token, dietary supplements, foods, and cosmetic compositions are readily contemplated.

As used herein, “prevention” or “preventing” of a disease, disorder, or condition refers to results that, in a statistical sample, exhibit a reduction in the occurrence of the disease, disorder, or condition in a sample or subject administered a therapeutic agent or agents relative to a control sample or subject. Such prevention is sometimes referred to as a prophylactic treatment.

As used herein, the term “simultaneous” therapeutic use refers to the administration of at least two active ingredients (e.g., therapeutic agents) by the same route and at the same time or at substantially the same time.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

As used herein, the terms “treating” or “treatment” refer to therapeutic treatment, wherein the object is to reduce, alleviate, or slow down (lessen) a pre-existing disease or disorder, or its related signs, symptoms, or conditions. By way of example, but not by way of limitation, a subject is successfully “treated” for a disease if, after receiving an effective amount of the composition/drug product or a pharmaceutically acceptable salt thereof, the subject shows observable and/or measurable reduction in or absence of one or more signs, symptoms, or conditions associated with the disease, disorder, or condition. It is also to be appreciated that the various modes of treatment of medical conditions as described are intended to mean “substantial,” which includes total alleviation of conditions, signs or symptoms of the disease or disorder, as well as “partial,” where some biologically or medically relevant result is achieved.

As used herein, the term “toxin” refers to a toxic chemical substance, the accumulation of or exposure to which causes detrimental effects in a subject. Toxic chemical substances comprise discrete small molecules, macromolecules, synthetic or natural products, amino acids, peptides, polypeptides, or proteins of any size, which are present systemically in a subject. Toxins can be identified or isolated from mixtures because they are discrete. As used herein, the term toxin excludes microorganisms and viruses, although the term includes toxins, if any, produced by a microorganism or a virus. Microorganisms can be bacteria, fungi, archaca, or protists. Microorganisms do not include viruses and prions, which are generally classified as non-living. It is believed that in certain instances, nucleotides, polynucleotides and nucleic acids, including ribonucleic acids (RNA) can be toxic; those molecules that are toxic are considered toxins herein. In some embodiments, a metabolite that accumulates to a level resulting in a toxic effect in a subject is a toxin. In one embodiment, toxins are present in the circulatory system of an affected subject. It is important to note that the term excludes toxic substances that are not systemic—that is, which do not circulate in the affected subject and are confined primarily to or build up in a specific tissue, such as pulmonary tissue.

### Pharmaceutical Compositions, Routes of Administration, and Dosing

The methods, uses, and compositions of the present application utilize a therapeutically effective amount of a composition to degrade or metabolize a systemic toxin in vivo. In some embodiments, a composition comprises an enzyme, or a polynucleotide encoding the same, that can be deposited in the lung tissue of a subject to degrade or metabolize a toxin that is capable of diffusing between lung tissue and the circulatory system of the subject. The deposition of such an enzyme or polynucleotide in lung tissue of the subject enhances the metabolic efficacy of the subject, and can result in relief from symptoms caused by a systemic toxin in the subject.

A pharmaceutical composition, as used herein, is a composition comprising a therapeutic agent and a carrier, inert or active, making the composition especially suitable for therapeutic use in vivo. A pharmaceutical composition can include, for example, an enzyme known to catalyze the breakdown or degradation of a toxin. A pharmaceutical composition can include, for example, a polynucleotide encoding an enzyme known to catalyze the breakdown or degradation of a toxin. In some embodiments, a pharmaceutical composition can include an enzyme known to catalyze the breakdown or degradation of a toxin, and a polynucleotide encoding said enzyme. In some preferred embodiments, a pharmaceutical composition is especially suitable for the delivery and deposition of a therapeutic agent (e.g., an enzyme or a polynucleotide encoding the same) to a tissue in a subject. In some preferred embodiments, a pharmaceutical composition is especially suitable for the delivery and deposition of an enzyme to lung tissue of a subject.

A pharmaceutical composition in accordance with the present disclosure may include an enzyme known to catalyze the breakdown or degradation of at least one systemic toxin. Non-limiting examples of enzymes known to catalyze the breakdown or degradation of a systemic toxin include phenylalanine ammonia lyase (PAL) (e.g., Genbank Accession No. NP_181241.1, NP_187645.1, NP_190894.1, NP_001190223.1, and NP_196043.2), phenylalanine hydroxylase (PAH) (e.g., Genbank Accession No. NP_000268 and NP_001341233), galactose degrading enzyme, galactose-1-phosphate uridylyltransferase (GALT) (e.g., Genbank Accession No. NP_000146.2 and NP_001245261.1), 2-oxoisovalerate dehydrogenase subunit alpha, mitochondrial (BCKDHA) (e.g., Genbank Accession No. NP_001158255.1 and NP_000700.1), 2-oxoisovalerate dehydrogenase subunit beta, mitochondrial (BCKDHB) (e.g., Genbank Accession No. NP_000047.1, NP_001305904.1, and NP_898871.1), BDT complex enzymes, kynureninase (e.g., Genbank Accession No. NP_001028170.1, NP_001186170.1, and NP_003928.1), cocaine esterase (e.g., Genbank Accession No. NP_001352334.1, NP_003860.3, NP_932327.2, NP_001352335.1, NP_001352336.1, and NP_001352337.1), arginase (e.g., Genbank Accession No. NP_001163.1, NP_000036.2, and NP_001231367.1), glutamine synthase (e.g., Genbank Accession No. NP_003866.1), arginosuccinate lyase (e.g., Genbank Accession No. NP_000039.2, NP_001020117.1, NP_001020114.1, NP_001020115.1), arginosuccinate synthase (e.g., Genbank Accession No. NP_000041.2 and NP_446464.1), carbamyl phosphate synthetase I (e.g., Genbank Accession No. NP_001116105.2, NP_001866.2), N-acetylglutamate synthetase (e.g., Genbank Accession No. NP_694551.1), ornithine transcarbamylase (e.g., Genbank Accession No. NP_000522.3 and NP_001394021.1), ornithine translocase (e.g., Genbank Accession No. NP_055067.1 and NP_114153.1), nicotine oxidoreductase, uricase (e.g., Genbank Accession No. NP_180191.1), acid alpha-glucosidase (GAA) (e.g., Genbank Accession No. NP_000143.2, NP_001073271.1, and NP_001073272.1), thiosulfate sulfurtransferase (rhodanese) (e.g., Genbank Accession No. NP_001257412.1 and NP_003303.2), collagenase (e.g., Genbank Accession No. NP_002412.1, NP_001291370.1, NP_001291371.1, NP_001291371.1), asparaginase (e.g., Genbank Accession No. NP_001077395.1 and NP_079356.3), anti-inhibitor coagulant complex, tissue-type plasminogen activator (t-Pa) (e.g., Alteplase; a tissue-type plasminogen activator), pegademase bovine (e.g., PEG-adenosine deaminase), alglucerase (e.g., a modified form of human β-glucocerebrosidase enzyme), imiglucerase (e.g., a form of recombinant human beta-glucocerebrosidase), Factor IX (e.g., Genbank Accession No. NP_000124.1 and NP_001300842.1), dnase, pancrelipase (e.g., amylase; lipase; protease), sacrosidase (e.g., a sucrase replacement enzyme), truncated (non-glycosylated) t-PA (357 of 527aa), coagulation Factor VIIa (e.g., Genbank Accession No. NP_000122.1, NP_001254483.1, NP_062562.1), tissue plasminogen activator, antihemophilic factor (AHF) (e.g., a coagulation activator), laronidase (e.g., a form of recombinant human alpha-L-iduronidase), agalsidase beta (e.g., a form of recombinant human alpha-galactosidase), hyaluronidase (e.g., an enzyme that reversibly depolymerizes hyaluronic acid), galsulfase (e.g., a variant form of the polymorphic human enzyme N-acetylgalactosamine 4-sulfatase), idursulfase (e.g., a purified lysosomal enzyme), alglucosidase alfa (e.g., an acid alpha-glucosidase (GAA) derivative), thrombin (e.g., a serine protease enzyme), velaglucerase alfa (e.g., a gene-activated human recombinant glucocerebrosidase), pegloticase (e.g., a pegylated, recombinant uricase), asparaginase, taliglucerase alfa (e.g., a recombinant glucocerebrosidase), plasmin (e.g., Genbank Accession No. NP_000292.1 and NP_001161810.1), carboxypeptidase g2 (e.g., UniProt Accession No. P06621), glucarpidase (e.g., a recombinant carboxypeptidase G2), coagulation Factor XIII A (e.g., Genbank Accession No. NP_000120.2), closulfase alfa (e.g., a synthetic version of the enzyme N-acetylgalactosamine-6-sulfatas), coagulation Factor X (e.g., Genbank Accession No. NP_001299604.1, NP_000495.1, and NP_001299603.1), asfotase alfa (e.g., recombinant glycoprotein that contains the catalytic domain (the active site) of tissue-nonspecific alkaline phosphatase), sebelipase alfa (e.g., a recombinant lysosomal acid lipase), cerliponase alfta (e.g., a hydrolytic lysosomal N-terminal tripeptidyl peptidase-1 (TPP1), vestronidase alfa-vjbk (e.g., a recombinant form of the human enzyme beta-glucuronidase), pegvaliase-pqpz (e.g., a recombinant phenylalanine ammonia lyase (PAL) enzyme), and combinations thereof. The enzyme can be an isolated human enzyme. The enzyme can be a non-human enzyme. The enzyme can be a recombinant enzyme. The enzyme can be a wildtype enzyme or a variant thereof. In some embodiments, a variant enzyme comprises an amino acid sequence having at least 65%, at least 70%, at least 75%, at least 80%, at least 85%, at least 90%, at least 95%, or at least 99% sequence identity to a corresponding wildtype enzyme.

In some embodiments, said enzyme, a variant thereof, or a combination thereof resides substantially in the subject's pulmonary tissue following introduction of the enzyme, a variant thereof, or a combination thereof. In some embodiments, said enzyme, a variant thereof, or a combination thereof is introduced under conditions that inhibit or do not support systemic delivery of said enzyme, variant thereof, or combination thereof to said subject. In some embodiments, said enzyme, a variant thereof, or a combination thereof is introduced to at least a portion of the lung of said subject in a manner that minimizes a systemic introduction into said subject. In some embodiments, said enzyme, a variant thereof, or a combination thereof is introduced in a manner that does not include pulmonary transmucosal delivery of said at least one preselected enzyme, a variant thereof, or a combination thereof to said subject.

In some embodiments, an enzyme known to enzymatically breakdown at least one toxin for use in accordance with technologies of the present disclosure is linked to a polypeptide, a polypeptide domain, or a fragment thereof that is absorbed into the lung by the receptor-mediated transcytosis pathway. Without wishing to be bound by any one theory, it is understood that such absorption by the receptor-mediated transcytosis pathway may increase the fraction of enzyme known to enzymatically breakdown at least one toxin residence in the subject's pulmonary tissue and/or minimizes a systemic introduction into said subject.

In some embodiments, an enzyme known to enzymatically breakdown at least one toxin for use in accordance with technologies of the present disclosure is PEGylated. Without wishing to be bound by any one theory, it is understood that enzyme PEGylation can reduce blood clearance of such enzymes (e.g., increasing the fraction of enzyme known to enzymatically breakdown at least one toxin residence in the subject's pulmonary tissue and/or minimizing a systemic introduction of said enzyme into said subject).

An enzyme used in accordance with the present disclosure, whether alone or included in a pharmaceutical composition, is preferably an enzyme that is capable of degrading or breaking down a systemic toxin that is capable of diffusing between the circulatory system and the lung tissue of a subject. Accordingly, a toxin that can be targeted by the methods of the present disclosure is a toxin that is capable of diffusing between the circulatory system and the lung tissue of a subject. Without wishing to be bound by theory, in some embodiments of the disclosure, certain upper limits of a size of a circulating toxin might be contemplated. Such limits could be, for example, 60 kDa and above, 100 kDa and above, 500 kDa and above, 1000 kDa and above, or 2000 kDa and above. Hence, in certain embodiments, toxins at or exceeding such limits might not be accessible to a degrading enzyme in a pulmonary context.

In some embodiments, a pharmaceutical composition can comprise one or more co-factors, co-enzymes, or co-substrates. In some embodiments, a pharmaceutical composition comprises at least one enzyme known to enzymatically breakdown at least one systemic toxin, and further comprises one or more co-factors, co-enzymes, co-substrates, or combinations thereof. Selection of the appropriate co-factor for use in accordance with an enzyme known to enzymatically breakdown at least one toxin is well within the level of one of ordinary skill in the art. Without wishing to be bound by any one theory, it is understood that the relative amount of reduced to oxidized cofactor can play an important role for the equilibrium of a biochemical reaction. Thus, regeneration of co-factors is important for metabolic efficiency. In some embodiments, pharmaceutical compositions of the present disclosure comprise an additional enzyme for co-factor regeneration.

A medicament is generally considered a composition or formulation specifically prepared for administration to a subject to address a disease, disorder, or condition (e.g., the presence of a systemic toxin, or the systemic accumulation of a toxic metabolite).

In some embodiments, the therapeutic agent(s) (e.g., an enzyme or a polynucleotide encoding an enzyme) can be formulated with little or no excipient or carrier. In some embodiments, the therapeutic agent(s) can be formulated such that the majority of the formulation is excipient or carrier. In brief, one of skill in the art will tailor the formulation to have a suitable amount of excipient or carrier based on the needs/condition of the subject, the kind and extent of the disease to be treated; the properties of the therapeutic agent or agents to be delivered and the selected mode of administration of the particular therapeutic agent or agents.

In certain embodiments, a pharmaceutical composition may further comprise at least one therapeutic agent other than the at least one enzyme known to enzymatically breakdown at least one toxin. For example, a pharmaceutical composition may further comprise one or more of anti-nausea agents, analgesic drugs, naltrexone, acamprosate, disulfiram, gabapentin, topiramate, antifungals, antibiotics, and probiotics. In some embodiments, a pharmaceutical composition may further comprise at least one agent that promotes enzyme absorption in the lungs in addition to the at least one enzyme known to enzymatically breakdown at least one toxin (or a polynucleotide encoding the same). Agents that may promote enzyme absorption in the lungs include, for example and without limitation, agents that promote transcytosis (e.g., receptor-mediated transcytosis), liposomes, cyclodextrins, and low molecular weight amino acids.

Pharmaceutical compositions may contain an effective amount of one or more of the therapeutic agent or agents as described herein and may optionally be disbursed (e.g. dissolved, suspended or otherwise) in a pharmaceutically acceptable carrier. The components of the pharmaceutical composition(s) may also be capable of being commingled with other therapeutic agents or active agents, and with each other, in a manner such that there is no interaction that would substantially impair the desired pharmaceutical efficiency.

As stated above, an “effective amount” refers to any amount of a particular therapeutic agent that is sufficient to achieve a desired biological effect. Combined with the teachings provided herein, by choosing among the various therapeutic agent(s) and weighing factors such as potency, relative bioavailability, patient body weight, severity of adverse side-effects and mode of administration, an effective prophylactic (i.e. preventative) or therapeutic treatment regimen can be planned which does not cause substantial unwanted toxicity and yet is effective to address the particular condition, disorder or disease of a particular subject in a therapeutic way. The effective amount of a therapeutic agent for any particular indication can vary depending on such factors as the disease, disorder or condition being treated, the particular agent(s) being administered, the size of the subject, the age of the subject, the overall health of the subject and/or the severity of the disease, disorder or condition. The effective amount may be determined during pre-clinical trials and/or clinical trials by methods familiar to physicians and clinicians. One of ordinary skill in the art can empirically determine the effective amount of a particular therapeutic agent or agents without necessitating undue experimentation. A maximum dose may be used, that is, the highest safe dose according to some medical judgment. Multiple doses per day may be contemplated to achieve appropriate systemic levels of administered agents. Appropriate systemic levels can be determined by, for example, measurement of the patient's peak or sustained plasma level of the drug. “Dose” and “dosage” are used interchangeably herein. A dose may be administered by oneself, by another or by way of a device (e.g., an inhaler or a nebulizer).

For any therapeutic composition described herein, the therapeutically effective amount can, for example, be initially determined from animal models. A therapeutically effective dose can also be determined from human data for agents which have been tested in humans and for agents which are known to exhibit similar pharmacological activities, such as other related active agents. Higher doses may be required for parenteral administration. The applied dose can be adjusted based on the relative bioavailability and potency of the administered agent. Adjusting the dose to achieve maximal efficacy based on the methods described above and other methods as are well-known in the art is well within the capabilities of the ordinarily skilled artisan.

Therapeutic agents (alone or as formulated in a pharmaceutical composition/medicament) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing of any animal model system known in the art can be used prior to administration to human subjects. In some embodiments, dosing can be tested directly in humans.

Dosage, toxicity and therapeutic efficacy of any therapeutic agents or compositions (e.g., formulations or medicaments comprising therapeutic compositions described herein), other/additional therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents that exhibit high therapeutic indices are advantageous.

An exemplary treatment regime can, for example, entail administration once per day, twice per day, thrice per day, once a week, or once a month. In therapeutic applications, a relatively high dosage at relatively short intervals is sometimes required until progression of the disease or condition is delayed, reduced, or terminated, or until the subject shows partial or complete amelioration of symptoms of a disease or condition. In some embodiments, a single administration is sufficient to delay, reduce, terminate, or ameliorate symptoms of a disease or condition.

For use in therapy, an effective amount of the therapeutic composition (alone or as formulated) can be administered to a subject by any mode that delivers the composition to the desired surface (e.g., lung tissue). Administering a pharmaceutical composition may be accomplished by any means known to the skilled artisan. Routes of administration include but are not limited to oral, topical, intranasal, intratracheal, inhalable, systemic, intravenous, subcutaneous, intraperitoneal, intradermal, intraocular, ophthalmical, intrathecal, intracerebroventricular, iontophoretical, transmucosal, intravitreal, or intramuscular administration. In some preferred embodiments, delivery of a composition is accomplished by inhalable administration. Administration includes self-administration, administration by another, and administration by a device (e.g., an inhaler or a nebulizer).

A therapeutic agent disclosed herein (e.g., enzymes, polypeptides, or a polynucleotides encoding the same) can be delivered to the subject in a formulation or medicament (i.e., a pharmaceutical composition). Formulations and medicaments can be prepared by, for example, dissolving or suspending a therapeutic agent disclosed herein (e.g., enzymes, polypeptides, polynucleotides encoding the same, and combinations thereof) in water, a solvent, a pharmaceutically acceptable carrier, salt, (e.g., NaCl or sodium phosphate), buffering agents, preservatives, compatible carriers, adjuvants, and optionally other therapeutically acceptable ingredients.

The pharmaceutical compositions (e.g. a formulation or medicament) can include a carrier (e.g., a pharmaceutically acceptable carrier), which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars (e.g., trehalose), polyalcohols such as mannitol, sorbitol, or sodium chloride in the composition.

The therapeutic agents or pharmaceutical compositions, when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. Pharmaceutical compositions may take such forms as suspensions, solutions or emulsions in oily or aqueous vehicles, and may contain formulatory agents such as suspending, stabilizing and/or dispersing agents. Additionally, suspensions of the therapeutic agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the therapeutic agents to allow for the preparation of highly concentrated solutions.

Systemic formulations include those designed for administration by injection, e.g., subcutaneous, intravenous, intramuscular, intrathecal, or intraperitoneal injection, as well as those designed for transdermal, transmucosal oral, or pulmonary administration.

For intravenous and other parenteral routes of administration, a composition can be formulated as a lyophilized preparation, as a lyophilized preparation of liposome-intercalated or lipid-encapsulated therapeutic agent(s), as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g., a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and may be preserved against the contaminating action of microorganisms such as bacteria and fungi.

Sterile injectable solutions (e.g., a formulation or medicament) can be prepared by incorporating the therapeutic agent(s) in the required amount in an appropriate solvent with one or a combination of ingredients enumerated above, as required, followed by filtered sterilization. Generally, dispersions are prepared by incorporating the therapeutic agent(s) into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

For oral administration, the agents can be formulated readily by combining the therapeutic agent(s) with pharmaceutically acceptable carriers well known in the art. Such carriers enable the therapeutic agent(s) to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above that may be chemically modified so that oral delivery of the derivative is efficacious. Generally, the chemical modification contemplated is the attachment of at least one moiety to the therapeutic agent(s), ingredient(s), and/or excipient(s), where said moiety permits (a) inhibition of acid hydrolysis; and (b) uptake into the blood stream from the stomach or intestine. Also desired is the increase in overall stability of the therapeutic agent(s), ingredient(s), and/or excipient(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of a therapeutic agent or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

Colorants and flavoring agents may all be included. For example, the therapeutic agent(s) or pharmaceutical composition(s) may be formulated and then further contained within an edible product, such as a refrigerated beverage containing colorants and flavoring agents.

One may dilute or increase the volume of the therapeutic agent(s) or pharmaceutical composition(s) with an inert material. These diluents could include carbohydrates, especially mannitol, lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic agent(s) or pharmaceutical composition(s) into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent(s) together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic agent(s).

An anti-frictional agent may be included in the formulation of the therapeutic agent(s) or pharmaceutical composition(s) to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic composition(s)/agent(s) or pharmaceutical composition(s) into the aqueous environment, a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation or medicament as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation or medicament disclosed herein or derivative either alone or as a mixture in different ratios.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the therapeutic agent(s) may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the therapeutic agent(s) or pharmaceutical composition(s) may be formulated as solutions, gels, ointments, creams, suspensions, etc. as are well-known in the art. Solutions, gels, ointments, creams or suspensions may be administered topically. The agents may also be formulated in rectal or vaginal compositions such as suppositories or retention enemas, e.g., containing conventional suppository bases such as cocoa butter or other glycerides.

For administration (introduction) by inhalation, therapeutic composition(s)/agent(s) or pharmaceutical composition(s) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament and/or therapeutic composition(s)/agent(s) can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol, the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic composition/agent and a suitable powder base such as lactose or starch. Alternatively, the therapeutic composition(s)/agent(s) or pharmaceutical composition(s) may be in powder form for constitution with a suitable vehicle, e.g., sterile pyrogen-free water or a suitable buffer, before use.

For administration (introduction) by inhalation, agent(s) (e.g., enzymes known to enzymatically breakdown at least one toxin) or composition(s) for use according to the present application may formulated for dry powder inhalation. Dry powder inhalation of macromolecules (e.g., enzymes of the present disclosure) can utilize a formulation comprising poly(ethylene glycol)-co-poly(glycerol-adipate-co-ω-pentadecalactone) as a biodegradable, polymer carrier, as described in Tawfeek H M, et al. (Int J Pharm. 2013 Jan. 30; 441 (1-2): 611-9; incorporated herein by reference in its entirety).

Nasal delivery of a therapeutic composition(s)/agent(s) or pharmaceutical composition(s) is also contemplated. Nasal delivery allows the passage of therapeutic composition(s)/agent(s) or pharmaceutical composition(s) to the blood stream directly after administering the therapeutic agent(s) or pharmaceutical composition(s) to the nose, without the necessity for deposition of the product in the lung. Formulations for nasal delivery include those with dextran or cyclodextran.

For nasal administration, one type of useful device is a small, hard bottle to which a metered dose sprayer is attached. In some embodiments, the metered dose is delivered by drawing a pharmaceutical composition (in solution form) into a chamber of defined volume, which chamber has an aperture dimensioned to aerosolize and aerosol formulation by forming a spray when a liquid in the chamber is compressed. The chamber is compressed to administer the therapeutic agent(s) or pharmaceutical composition(s). In a specific embodiment, the chamber is a piston arrangement. Such devices are commercially available.

Alternatively, a plastic squeeze bottle with an aperture or opening dimensioned to acrosolize an aerosol formulation by forming a spray when squeezed can be used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic agent(s) or pharmaceutical composition(s).

Contemplated for use in the practice of this technology are a wide range of mechanical devices designed for inhalable delivery of therapeutic products, including but not limited to nebulizers, metered dose inhalers, and powder inhalers, all of which are familiar to those skilled in the art.

Some specific examples of commercially available devices suitable for the practice of this technology are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of the therapeutic composition(s)/agent(s) or pharmaceutical composition(s). Typically, each formulation is specific to the type of device employed and may involve the use of an appropriate propellant material, in addition to the usual diluents, adjuvants and/or carriers useful in therapy. Also, the use of liposomes, microcapsules, microspheres, nanoparticles, nanospheres, inclusion complexes, or other types of carriers is contemplated.

Formulations suitable for use with a nebulizer, either jet or ultrasonic, can, for example, comprise therapeutic composition(s)/agent(s) or pharmaceutical composition(s) dissolved in water at a concentration of about 0.01 to 50 mg of biologically active composition per mL of solution. The formulation may also include a buffer and optionally a simple sugar (e.g., for inhibitor stabilization and regulation of osmotic pressure). The nebulizer formulation may also contain a surfactant, to reduce or prevent surface induced aggregation of the therapeutic agent(s) or pharmaceutical composition(s) disclosed herein caused by atomization of the solution in forming the aerosol.

Formulations for use with a metered-dose inhaler device may generally comprise a finely divided powder comprising the therapeutic composition(s)/agent(s) or pharmaceutical composition(s) disclosed herein suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device may comprise a finely divided dry powder containing the therapeutic composition(s)/agent(s) or pharmaceutical composition(s) and may also include a bulking agent, such as lactose, sorbitol, sucrose, or mannitol in amounts which facilitate dispersal of the powder from the device, e.g., 50 to 90% by weight of the formulation. The therapeutic agent(s)/pharmaceutical composition(s) can advantageously be prepared in particulate or nanoparticulate form with an average particle size of less than 10 micrometers (μm), most preferably 0.5 to 5 μm, for most effective delivery to the deep lung.

### Formulations and Medicaments

Any of the formulations (which can also be referred to as a medicament or composition when formulated for administration to a subject having a certain affliction or medical condition that requires medical attention) described in the section above entitled: “Pharmaceutical Compositions, Routes of Administration, and Dosing” can be applied to produce a composition (i.e. a formulation or medicament) suitable for administration to a subject in need thereof. Thus, in some embodiments, this application is directed to compositions, formulations, and medicaments suitable for administration to a subject suffering from, or believed to be suffering from, a disease or condition in which a systemic toxin (e.g., an exogenous or foreign toxin, or an endogenous toxin (e.g., an accumulated metabolite)) is exerting a negative impact on the subject.

### Therapeutic Methods and In Vivo Toxin Degradation

Toxins that exert negative effects on a subject can enter the systemic circulation, causing them to act on numerous systems of the body. It is understood that such systemic toxins can be transported from the systemic circulation to the pulmonary circulation, where they can readily diffuse from the pulmonary blood supply into the lung tissue. Without wishing to be bound by any one theory, it is understood that introduction of compositions comprising at least one enzyme known to enzymatically breakdown at least one systemic toxin to at least a portion of a lung of a subject using methods described herein can facilitate in vivo degradation of the systemic toxin, effectively converting the lungs to a tunable metabolic organ with expanded metabolic capacities introduced by such exogenously added enzymes.

The present disclosure provides methods of alleviating one or more negative effects of a toxin in vivo. In some embodiments, a method of alleviating one or more negative effects of a toxin in vivo comprises introducing at least one preselected enzyme to pulmonary tissue of a subject, preferably an enzyme known to enzymatically breakdown at least one toxin that is exerting one or more negative effects against said subject.

In some embodiments, a method of alleviating one or more negative effects of a toxin in vivo comprises allowing the enzymatic breakdown of at least one toxin to take place in vivo, alleviating said one or more negative effects.

In some embodiments, a method of alleviating one or more negative effects of a toxin in vivo comprises (a) introducing at least one preselected enzyme to pulmonary tissue of a subject, said at least one preselected enzyme being known to enzymatically breakdown at least one toxin that is exerting one or more negative effects against said subject; and (b) allowing the enzymatic breakdown of said at least one toxin to take place in vivo, alleviating said one or more negative effects.

The introducing step can be accomplished by any means that results in contacting pulmonary tissue of the subject with either a polypeptide having an amino acid sequence corresponding to said at least one preselected enzyme, a polynucleotide encoding said at least one polypeptide, or both the polypeptide and the polynucleotide encoding the polypeptide. In some embodiments, the introducing step is accomplished by transducing a plurality of cells present in pulmonary tissue to express said at least one preselected enzyme. In some embodiments, the plurality of cells is transduced with DNA or a construct thereof. In some embodiments, the plurality of cells is transduced with mRNA or a construct thereof. The DNA, mRNA, or constructs thereof may be formulated with or without a pharmaceutically acceptable carrier.

In some embodiments, which said introducing step comprises contacting pulmonary tissue with either a polypeptide having an amino acid sequence corresponding to said at least one preselected enzyme, a polynucleotide encoding said at least one polypeptide, or both

In some embodiments, a subject is afflicted with one or more genetic disorders. In some preferred embodiments, the subject is afflicted with a genetic disorder that results in a condition involving a systemic accumulation of a toxin (e.g., a toxic metabolite). In some embodiments, the condition is a result of a deficiency in an enzyme that, in a subject who does not have the condition, catalyzes the breakdown of or conversion of a biological macromolecule or metabolite. In some instances, the toxin is a metabolite that exerts a toxic effect only when present systemically in an amount sufficient to exert a negative effect on the subject, but is otherwise non-toxic when present systemically at a level maintained by one or more metabolizing enzyme. Non-limiting examples of genetic disorders and conditions that involve a toxic and systemic accumulation of a toxin (e.g., metabolite) include phenylketonuria, gout, cystinuria, ornithine transcarbamylase deficiency (OTCD), galactosemia, maple syrup urine disease, tumor suppression disorder, cocaine use disorder, urea cycle disorder, tobacco use disorder, Pompe's disease, sucrase isomaltase deficiency, arginase deficiency, and hyperargininemia. In some embodiments, one or more genetic disorders is selected from the group consisting of phenylketonuria, gout, cystinuria, ornithine transcarbamylase deficiency (OTCD), galactosemia, maple syrup urine disease, tumor suppression disorder, cocaine use disorder, urea cycle disorder, tobacco use disorder, Pompe's disease, sucrase isomaltase deficiency, arginase deficiency, hyperargininemia, and combinations thereof. A genetic disorder addressable by the methods of the present disclosure is preferably a genetic disorder that prevents a breakdown of at least one toxin.

In some embodiments, a subject has been exposed to at least one toxin. Non-limiting examples of toxins to which a subject may be exposed include phenylalanine, uric acid, cystine, arginine, lysine, ornithine, leucine, isoleucine, valine, an amino acid, galactose, kynurenine, cocaine, ammonia, nicotine, cyanide, and organophosphates. In some embodiments, at least one toxin is selected from the group consisting of phenylalanine, uric acid, cystine, arginine, lysine, ornithine, leucine, isoleucine, valine, an amino acid, galactose, kynurenine, cocaine, ammonia, nicotine, cyanide, organophosphates, and combinations thereof.

An enzyme for use according to the present disclosure can include an enzyme known to catalyze the breakdown or degradation of a toxin. Non-limiting examples of enzymes known to catalyze the breakdown or degradation of a toxin include phenylalanine ammonia lyase (PAL), phenylalanine hydroxylase (PAH), galactose degrading enzyme, galactose-1-phosphate uridylyltransferase (GALT), 2-oxoisovalerate dehydrogenase subunit alpha, mitochondrial (BCKDHA), 2-oxoisovalerate dehydrogenase subunit beta, mitochondrial (BCKDHB), BDT complex enzymes, kynureninase, cocaine esterase, arginase, glutamine synthase, arginosuccinate lyase, arginosuccinate synthase, carbamyl phosphate synthetase I, N-acetylglutamate synthetase, ornithine transcarbamylase, ornithine translocase, nicotine oxidoreductase, uricase. In some embodiments of methods described herein, a preselected enzyme is selected from the group consisting of phenylalanine ammonia lyase (PAL), phenylalanine hydroxylase (PAH), galactose degrading enzyme, galactose-1-phosphate uridylyltransferase (GALT), 2-oxoisovalerate dehydrogenase subunit alpha, mitochondrial (BCKDHA), 2-oxoisovalerate dehydrogenase subunit beta, mitochondrial (BCKDHB), BDT complex enzymes, kynureninase, cocaine esterase, arginase, glutamine synthase, arginosuccinate lyase, arginosuccinate synthase, carbamyl phosphate synthetase I, N-acetylglutamate synthetase, ornithine transcarbamylase, ornithine translocase, nicotine oxidoreductase, uricase, acid alpha-glucosidase (GAA), and combinations thereof. Additional examples of enzymes known to catalyze the breakdown or degradation of a toxin include collagenase, asparaginase, anti-inhibitor coagulant complex, tissue-type plasminogen activator (t-Pa), Alteplase, pegademase bovine, alglucerase, imiglucerase, Factor IX, dnase, pancrelipase (amylase; lipase; protease), sacrosidase, truncated (non-glycosylated) tPA (357 of 527aa), coagulation Factor VIIa, recombinant, tissue plasminogen activator variant, uricase (Aspergillus flavus), antihemophilic factor, laronidase, agalsidase beta, hyaluronidase (ovine), hyaluronidase (bovine), galsulfase, hyaluronidase (human), idursulfase, alglucosidase alfa, thrombin (human), thrombin (Bovine), velaglucerase alfa, pegloticase, asparaginase (Erwinia chrysanthem), taliglucerase alfa, recombinant truncated form of human plasmin, recombinant carboxypeptidase g2, glucarpidase, coagulation Factor XIII A, elosulfase alfa, coagulation Factor X, asfotase alfa, sebelipase alfa, cerliponase alfta, vestronidase alfa-vjbk, pegvaliase-pqpz, and combinations thereof.

A method of alleviating one or more negative effects of at least one toxin in vivo can also be applied to treat a genetic disorder in a subject, preferably a genetic disorder in which the natural breakdown of one or more toxins is impaired in the subject. For example, in some embodiments, a method of alleviating one or more negative effects of at least one toxin in vivo can also be applied to treat a genetic disorder in a subject that has a genetic condition, such as an enzyme deficiency, leading to accumulation of an endogenous metabolite. Non-limiting examples of genetic conditions involving an enzyme deficiency include phenylketonuria, hyperuricemia, gout, cystinuria, ornithine transcarbamylase deficiency (OTCD), galactosemia, maple syrup urine disease, and urea cycle disorder. In some embodiments, a method of alleviating one or more negative effects of at least one toxin in vivo can be practiced in a subject suffering from a condition involving secondary accumulation of an endogenous metabolite. In some embodiments, a method of alleviating one or more negative effects of at least one toxin in vivo can be practiced in a subject suffering from an autoimmune disease. In some embodiments, a method of alleviating one or more negative effects of at least one toxin in vivo can be practiced in a subject suffering from a cancer, such as leukemia. In some embodiments, methods of the present disclosure can be practiced in a subject suffering from a substance use disorder. Non-limiting examples of substance use disorders include cocaine use disorder, tobacco use disorder, nicotine use disorder, stimulant use disorder, sedative use disorder, and opioid use disorder.

A method of alleviating one or more negative effects of at least one toxin in vivo can also be applied to treat a subject who has that has consumed or otherwise come into contact with a toxin, and said toxin has entered the subject's systemic circulation. In some embodiments, methods of the present disclosure can be practiced in a subject that has been poisoned. In some embodiments, methods of the present disclosure can be practiced in a subject contemplating a consumption of, is in a process of consuming, or has consumed one or more solid or liquid preparations comprising a toxin, such as a drug or other toxic agent. For example, in some embodiments, a method of alleviating one or more negative effects of at least one toxin in vivo can be practiced in a subject that has consumed or otherwise come into contact with cocaine, and said cocaine has entered the subject's systemic circulation. In some embodiments, a method of alleviating one or more negative effects of at least one toxin in vivo can be practiced in a subject that has consumed or otherwise come into contact with cyanide, and said cyanide has entered the subject's systemic circulation.

The present disclosure provides a method of alleviating one or more negative effects of leukemia in a subject. In some embodiments, a method of alleviating one or more negative effects of leukemia comprises introducing asparaginase to pulmonary tissue of a subject diagnosed with or suspected of having leukemia, said asparaginase being known to enzymatically break down asparagine. In some embodiments, the asparaginase is L-asparaginase. In some embodiments, a method of alleviating one or more negative effects of leukemia comprises allowing the enzymatic breakdown of asparagine to take place in vivo, alleviating said one or more negative effects. Without wishing to be bound by any one theory, it is understood that in at least some forms of leukemia, leukemic cells are unable to synthesize L-asparagine, and thus such leukemic cells rely on systemic L-asparagine to survive. The methods disclosed herein result in the breakdown of asparagine, which can serve to deprive leukemic cells of nutrients required for survival, alleviating one or more negative effects on a subject having leukemic cells. In some embodiments, a method of alleviating one or more negative effects of leukemia comprises (a) introducing asparaginase to pulmonary tissue of a subject; and (b) allowing the enzymatic breakdown of asparagine to take place in vivo, alleviating said one or more negative effects. In some embodiments, the leukemia comprises acute lymphoblastic leukemia (ALL).

The present disclosure provides a method of alleviating one or more negative effects of excess oxalate in a subject. In some embodiments, a method of alleviating one or more negative effects of excess oxalate comprises introducing oxalate decarboxylase to pulmonary tissue of a subject diagnosed with or suspected of having oxalosis, primary hyperoxaluria, or secondary hyperoxaluria, said oxalate decarboxylase being known to enzymatically break down oxalate. In some embodiments, a method of alleviating one or more negative effects of oxalosis, primary hyperoxaluria, or secondary hyperoxaluria comprises allowing the enzymatic breakdown of oxalate to take place in vivo, alleviating said one or more negative effects. Without wishing to be bound by any one theory, it is understood that oxalosis can occur due to impaired kidney function, including in patients who have primary and intestine-related causes of hyperoxaluria, resulting in the accumulation of oxalate in the blood. The methods disclosed herein result in the breakdown of oxalate, which can serve to reduce circulating oxalate levels, alleviating one or more negative effects of oxalosis, primary hyperoxaluria, or secondary hyperoxaluria in a subject. In some embodiments, a method of alleviating one or more negative effects of oxalosis comprises (a) introducing oxalate decarboxylase to pulmonary tissue of a subject; and (b) allowing the enzymatic breakdown of oxalate to take place in vivo, alleviating said one or more negative effects. In some embodiments, the oxalate is endogenously produced oxalate. In some embodiments, the oxalate is dietary oxalate.

The present disclosure provides a method of alleviating one or more negative effects of phenylketonuria in a subject. In some embodiments, a method of alleviating one or more negative effects of phenylketonuria comprises introducing phenylalanine ammonia lyase (PAL) to pulmonary tissue of a subject diagnosed with or suspected of having phenylketonuria, said PAL being known to enzymatically breakdown phenylalanine. In some embodiments, a method of alleviating one or more negative effects of phenylketonuria comprises allowing the enzymatic breakdown of phenylalanine to take place in vivo, alleviating said one or more negative effects. Without wishing to be bound by any one theory, it is understood that phenylketonuria can occur due to loss of function of the phenylalanine hydroxylase (PAH) gene, resulting in the accumulation of phenylalanine in the blood. The methods disclosed herein result in the breakdown of phenylalanine, which can serve to reduce circulating phenylalanine levels, alleviating one or more negative effects of phenylketonuria in a subject. In some embodiments, a method of alleviating one or more negative effects of phenylketonuria comprises (a) introducing PAL to pulmonary tissue of a subject; and (b) allowing the enzymatic breakdown of phenylalanine to take place in vivo, alleviating said one or more negative effects.

The present disclosure provides a method of alleviating one or more negative effects of hyperuricemia in a subject. In some embodiments, a method of alleviating one or more negative effects of hyperuricemia comprises introducing uricase to pulmonary tissue of a subject diagnosed with or suspected of having hyperuricemia, said uricase being known to enzymatically breakdown uric acid. In some embodiments, a method of alleviating one or more negative effects of hyperuricemia comprises allowing the enzymatic breakdown of uric acid to take place in vivo, alleviating said one or more negative effects. Without wishing to be bound by any one theory, it is understood that hyperuricemia can occur due to underexcretion or overproduction of uric acid, resulting in the accumulation of uric acid in the blood. Untreated hyperuricemia can impair kidney function and result in gout. The methods disclosed herein result in the breakdown of uric acid, which can serve to reduce circulating uric acid levels, alleviating one or more negative effects of hyperuricemia in a subject. In some embodiments, a method of alleviating one or more negative effects of hyperuricemia comprises (a) introducing uricase to pulmonary tissue of a subject; and (b) allowing the enzymatic breakdown of uric acid to take place in vivo, alleviating said one or more negative effects.

The present disclosure provides a method of alleviating one or more negative effects of ornithine transcarbamylase deficiency (OTCD) in a subject. In some embodiments, a method of alleviating one or more negative effects of ornithine transcarbamylase deficiency comprises introducing ornithine transcarbamylase to pulmonary tissue of a subject diagnosed with or suspected of having ornithine transcarbamylase deficiency, said ornithine transcarbamylase being known to enzymatically breakdown ammonia. In some embodiments, a method of alleviating one or more negative effects of ornithine transcarbamylase deficiency comprises allowing the enzymatic breakdown of ammonia to take place in vivo, alleviating said one or more negative effects. Without wishing to be bound by any one theory, it is understood that ornithine transcarbamylase deficiency can result in the accumulation of ammonia in the blood. The methods disclosed herein result in the breakdown of ammonia, which can serve to reduce circulating ammonia levels, alleviating one or more negative effects of ornithine transcarbamylase deficiency in a subject. In some embodiments, a method of alleviating one or more negative effects of ornithine transcarbamylase deficiency comprises (a) introducing ornithine transcarbamylase to pulmonary tissue of a subject; and (b) allowing the enzymatic breakdown of ammonia to take place in vivo, alleviating said one or more negative effects.

In some embodiments, a method of alleviating one or more negative effects of ornithine transcarbamylase deficiency (OTCD) comprises introducing glutamine synthase to pulmonary tissue of a subject diagnosed with or suspected of having ornithine transcarbamylase deficiency, said glutamine synthase being known to enzymatically remove ammonia. In some embodiments, a method of alleviating one or more negative effects of ornithine transcarbamylase deficiency comprises allowing the enzymatic removal of ammonia to take place in vivo, alleviating said one or more negative effects. Without wishing to be bound by any one theory, it is understood that ornithine transcarbamylase deficiency can result in the accumulation of ammonia in the blood. The methods disclosed herein result in the removal of ammonia, which can serve to reduce circulating ammonia levels, alleviating one or more negative effects of ornithine transcarbamylase deficiency in a subject. In some embodiments, a method of alleviating one or more negative effects of ornithine transcarbamylase deficiency comprises (a) introducing glutamine synthase to pulmonary tissue of a subject; and (b) allowing the enzymatic removal of ammonia to take place in vivo, alleviating said one or more negative effects.

The present disclosure provides a method of alleviating one or more negative effects of hyperammonemia in a subject. In some embodiments, a method of alleviating one or more negative effects of hyperammonemia comprises introducing glutamine synthase to pulmonary tissue of a subject diagnosed with or suspected of having hyperammonemia, said glutamine synthase being known to enzymatically remove ammonia. In some embodiments, a method of alleviating one or more negative effects of hyperammonemia comprises allowing the enzymatic removal of ammonia to take place in vivo, alleviating said one or more negative effects. The methods disclosed herein result in the removal of ammonia, which can serve to reduce circulating ammonia levels, alleviating one or more negative effects of hyperammonemia in a subject. In some embodiments, a method of alleviating one or more negative effects of hyperammonemia comprises (a) introducing glutamine synthase to pulmonary tissue of a subject; and (b) allowing the enzymatic removal of ammonia to take place in vivo, alleviating said one or more negative effects.

The present disclosure provides a method of alleviating one or more negative effects of galactosemia in a subject. In some embodiments, a method of alleviating one or more negative effects of galactosemia comprises introducing galactose-1-phosphate uridylyltransferase (GALT) to pulmonary tissue of a subject diagnosed with or suspected of having galactosemia, said GALT being known to enzymatically breakdown galactose. In some embodiments, a method of alleviating one or more negative effects of galactosemia comprises allowing the enzymatic breakdown of galactose to take place in vivo, alleviating said one or more negative effects. Without wishing to be bound by any one theory, it is understood that galactosemia can result in the accumulation of galactose in the blood. The methods disclosed herein result in the breakdown of galactose, which can serve to reduce circulating galactose levels, alleviating one or more negative effects of galactosemia in a subject. In some embodiments, a method of alleviating one or more negative effects of galactosemia comprises (a) introducing GALT to pulmonary tissue of a subject; and (b) allowing the enzymatic breakdown of galactose to take place in vivo, alleviating said one or more negative effects.

The present disclosure provides a method of alleviating one or more negative effects of Pompe's disease in a subject. In some embodiments, a method of alleviating one or more negative effects of Pompe's disease comprises introducing acid alpha-glucosidase (GAA) to pulmonary tissue of a subject diagnosed with or suspected of having Pompe's disease, said GAA being known to enzymatically breakdown glycogen. In some embodiments, a method of alleviating one or more negative effects of Pompe's disease comprises allowing the enzymatic breakdown of glycogen to take place in vivo, alleviating said one or more negative effects. Without wishing to be bound by any one theory, it is understood that Pompe's disease can result in the accumulation of glycogen in the body. The methods disclosed herein result in the breakdown of glycogen, which can serve to reduce glycogen levels, alleviating one or more negative effects of Pompe's disease in a subject. In some embodiments, a method of alleviating one or more negative effects of Pompe's disease comprises (a) introducing GAA to pulmonary tissue of a subject; and (b) allowing the enzymatic breakdown of glycogen to take place in vivo, alleviating said one or more negative effects.

The present disclosure provides a method of alleviating one or more negative effects of sucrase isomaltase deficiency in a subject. In some embodiments, a method of alleviating one or more negative effects of sucrase isomaltase deficiency comprises introducing sucrase isomaltase to pulmonary tissue of a subject diagnosed with or suspected of having sucrase isomaltase deficiency, said sucrase isomaltase being known to enzymatically breakdown di- and oligosaccharides. In some embodiments, a method of alleviating one or more negative effects of sucrase isomaltase deficiency comprises allowing the enzymatic breakdown of di- and oligosaccharides to take place in vivo, alleviating said one or more negative effects. Without wishing to be bound by any one theory, it is understood that sucrase isomaltase deficiency can result in the accumulation of di- and oligosaccharides in the blood. The methods disclosed herein result in the breakdown of di- and oligosaccharides, which can serve to reduce di- and oligosaccharides levels in the blood, alleviating one or more negative effects of sucrase isomaltase deficiency in a subject. In some embodiments, a method of alleviating one or more negative effects of sucrase isomaltase deficiency comprises (a) introducing sucrase isomaltase to pulmonary tissue of a subject; and (b) allowing the enzymatic breakdown of di- and oligosaccharides to take place in vivo, alleviating said one or more negative effects.

The present disclosure provides a method of alleviating one or more negative effects of arginase deficiency in a subject. In some embodiments, a method of alleviating one or more negative effects of arginase deficiency comprises introducing arginase to pulmonary tissue of a subject diagnosed with or suspected of having arginase deficiency, said arginase being known to enzymatically breakdown arginine. In some embodiments, a method of alleviating one or more negative effects of arginase deficiency comprises allowing the enzymatic breakdown of arginine to take place in vivo, alleviating said one or more negative effects. Without wishing to be bound by any one theory, it is understood that arginase deficiency can result in the accumulation of arginine in the blood. The methods disclosed herein result in the breakdown of arginine, which can serve to reduce arginine levels in the blood, alleviating one or more negative effects of arginase deficiency in a subject. In some embodiments, a method of alleviating one or more negative effects of arginase deficiency comprises (a) introducing arginase to pulmonary tissue of a subject; and (b) allowing the enzymatic breakdown of arginine to take place in vivo, alleviating said one or more negative effects.

The present disclosure provides a method of alleviating one or more negative effects of hyperargininemia in a subject. In some embodiments, a method of alleviating one or more negative effects of hyperargininemia comprises introducing arginase to pulmonary tissue of a subject diagnosed with or suspected of having hyperargininemia, said arginase being known to enzymatically breakdown arginine. In some embodiments, a method of alleviating one or more negative effects of hyperargininemia comprises allowing the enzymatic breakdown of arginine to take place in vivo, alleviating said one or more negative effects. The methods disclosed herein result in the breakdown of arginine, which can serve to reduce arginine levels in the blood, alleviating one or more negative effects of hyperargininemia in a subject. In some embodiments, a method of alleviating one or more negative effects of hyperargininemia comprises (a) introducing arginase to pulmonary tissue of a subject; and (b) allowing the enzymatic breakdown of arginine to take place in vivo, alleviating said one or more negative effects.

The present disclosure provides a method of alleviating one or more negative effects of cyanide poisoning in a subject. In some embodiments, a method of alleviating one or more negative effects of cyanide poisoning comprises introducing thiosulfate sulfurtransferase (rhodanese) to pulmonary tissue of a subject diagnosed with or suspected of having cyanide poisoning. In some embodiments, a method of alleviating one or more negative effects of cyanide poisoning comprises allowing the enzymatic breakdown of cyanide to take place in vivo, alleviating said one or more negative effects. Without wishing to be bound by any one theory, it is understood that cyanide poisoning can occur due to the ingestion of cyanide salts, drinking pure liquid prussic acid, skin absorption of prussic acid, intravenous infusion of nitroprusside for hypertensive crisis, or the inhalation of hydrogen cyanide gas, resulting in the accumulation of cyanide in the blood. The methods disclosed herein result in the breakdown of cyanide, which can serve to reduce circulating cyanide levels, alleviating one or more negative effects of cyanide poisoning in a subject. In some embodiments, a method of alleviating one or more negative effects of cyanide poisoning comprises (a) introducing rhodanese to pulmonary tissue of a subject; and (b) allowing the enzymatic breakdown of cyanide to take place in vivo, alleviating said one or more negative effects. In some embodiments, the rhodanese can be introduced in conjunction with a co-factor, such as sodium thiosulfate, to pulmonary tissue of the subject.

In some embodiments, allowing the enzymatic breakdown or degradation of a toxin comprises allowing the enzymatic breakdown or degradation of at least one toxin that has diffused or migrated from the subject's circulatory system into the subject's lung and/or lung mucous to take place in vivo. In some embodiments, the enzymatic breakdown or degradation of a toxin is facilitated in a subject's lung and/or lung mucous. In some embodiments, the enzymatic breakdown or degradation of a toxin is facilitated in a subject's lung and/or lung mucous, in vivo.

Methods of the present disclosure can reduce the level of a toxin (e.g., a toxic metabolite, endogenous molecule, or consumed substance) in the blood of the subject. For example, the level of a toxin in the blood of the subject can be reduced by about 10% or more, about 15% or more, about 20% or more, about 25% or more, about 30% or more, about 40% or more, about 50% or more, about 60% or more, about 75% or more, about 90% or more, about 95% or more, or about 99% or more, relative to the level present before application of one or more of the technologies disclosed herein. In some embodiments, the level of a toxin in the blood of the subject can be reduced by about 10% or more, about 15% or more, about 20% or more, about 25% or more, about 30% or more, about 40% or more, about 50% or more, about 60% or more, about 75% or more, about 90% or more at a time about one hour, about two hours, about four hours, about 6 hours, about 10 hours, about 12 hours, about 18 hours, about 24 hours, about 36 hours, about 48 hours, about 72 hours, or about 96 hours or more after introducing a composition comprising at least one enzyme known to enzymatically breakdown at least one toxin to pulmonary tissue of the subject, wherein the reduction is relative to the level present before application of one or more of the technologies disclosed herein. In some embodiments, the level of at toxin in the blood of the subject can be reduced by about 10% or more, about 25% or more, about 50% or more, about 75% or more, about 90% or more, about 95% or more, or about 99% or more at a time about one hour after introducing a composition comprising at least one enzyme known to enzymatically breakdown at least one toxin to pulmonary tissue of the subject, wherein the reduction is relative to the level present before application of one or more of the technologies disclosed herein.

Technologies to measure blood toxin levels in subject are readily known in the art and their use in accordance with the present disclosure is well within the level of one of ordinary skill in the art.

The present disclosure provides a method of transforming a lung of a subject to include at least one enzymatic breakdown function comprising introducing to a lung of a subject at least one preselected enzyme or a polynucleotide encoding at least one preselected enzyme known to facilitate an enzymatic breakdown of at least one toxin that, if present, is present systemically in the subject. Transformation can be accomplished by depositing at least one enzyme in the pulmonary tissue (i.e., lung tissue) of a subject, or by transducing cells of the lung tissue to express the at least one enzyme.

The present disclosure provides a transformed lung capable of enzymatically breaking down at least one toxin that is systemically present in a subject, including the subject's lung, comprising pulmonary tissue transformed to harbor at least one preselected enzyme known to enzymatically breakdown the at least one toxin. In some embodiments, the transformed pulmonary tissue harbors at least one preselected enzyme via an introduction of either a polypeptide having an amino acid sequence corresponding to said preselected enzyme, a polynucleotide encoding said polypeptide, or both. Methods of transforming tissues by introducing an enzyme or polynucleotide encoding the same are well known in the art. A transformed lung of the present disclosure can be produced by one or more transformation methods known in the art. In some embodiments, a transformed lung comprises at least a portion of a lung comprising an exogenously introduced enzyme, or a polynucleotide encoding the same. In some embodiments, a transformed lung comprises an exogenously introduced enzyme selected from the group consisting of phenylalanine ammonia lyase (PAL), phenylalanine hydroxylase (PAH), galactose degrading enzyme, galactose-1-phosphate uridylyltransferase (GALT), 2-oxoisovalerate dehydrogenase subunit alpha, mitochondrial (BCKDHA), 2-oxoisovalerate dehydrogenase subunit beta, mitochondrial (BCKDHB), BDT complex enzymes, kynureninase, cocaine esterase, arginase, glutamine synthase, arginosuccinate lyase, arginosuccinate synthase, carbamyl phosphate synthetase I, N-acetylglutamate synthetase, ornithine transcarbamylase, ornithine translocase, nicotine oxidoreductase, uricase, acid alpha-glucosidase (GAA), thiosulfate sulfurtransferase (rhodanese), collagenase, asparaginase, anti-inhibitor coagulant complex, tissue-type plasminogen activator (t-Pa), Alteplase, pegademase bovine, alglucerase, imiglucerase, Factor IX, dnase, pancrelipase (amylase; lipase; protease), sacrosidase, truncated (non-glycosylated) t-PA (357 of 527aa), coagulation Factor VIIa, tissue plasminogen activator, antihemophilic factor (AHF), laronidase, agalsidase beta, hyaluronidase, galsulfase, idursulfase, alglucosidase alfa, thrombin, velaglucerase alfa, pegloticase, asparaginase, taliglucerase alfa, plasmin, carboxypeptidase g2, glucarpidase, coagulation Factor XIII A, closulfase alfa, coagulation Factor X, asfotase alfa, sebelipase alfa, cerliponase alfa, vestronidase alfa-vjbk, pegvaliase-pqpz, and combinations thereof, or a polynucleotide encoding the same.

The present disclosure provides a transformed pulmonary cell comprising an alveolar cell harboring at least one preselected enzyme known to enzymatically breakdown at least one toxin. In some embodiments, a transformed alveolar cell harbors at least one preselected enzyme via an introduction of either a polypeptide having an amino acid sequence corresponding to said preselected enzyme, a polynucleotide encoding said polypeptide, or both. Methods of transforming pulmonary cells by introducing an enzyme or polynucleotide encoding the same are well known in the art. A transformed pulmonary cell of the present disclosure can be produced by one or more transformation methods known in the art. In some embodiments, a transformed pulmonary cell comprises an alveolar cell comprising an exogenously introduced enzyme, or a polynucleotide encoding the same. In some embodiments, a transformed pulmonary cell comprises an exogenously introduced enzyme selected from the group consisting of phenylalanine ammonia lyase (PAL), phenylalanine hydroxylase (PAH), galactose degrading enzyme, galactose-1-phosphate uridylyltransferase (GALT), 2-oxoisovalerate dehydrogenase subunit alpha, mitochondrial (BCKDHA), 2-oxoisovalerate dehydrogenase subunit beta, mitochondrial (BCKDHB), BDT complex enzymes, kynureninase, cocaine esterase, arginase, glutamine synthase, arginosuccinate lyase, arginosuccinate synthase, carbamyl phosphate synthetase I, N-acetylglutamate synthetase, ornithine transcarbamylase, ornithine translocase, nicotine oxidoreductase, uricase, acid alpha-glucosidase (GAA), thiosulfate sulfurtransferase (rhodanese), collagenase, asparaginase, anti-inhibitor coagulant complex, tissue-type plasminogen activator (t-Pa), Alteplase, pegademase bovine, alglucerase, imiglucerase, Factor IX, dnase, pancrelipase (amylase; lipase; protease), sacrosidase, truncated (non-glycosylated) t-PA (357 of 527aa), coagulation Factor VIIa, tissue plasminogen activator, antihemophilic factor (AHF), laronidase, agalsidase beta, hyaluronidase, galsulfase, idursulfase, alglucosidase alfa, thrombin, velaglucerase alfa, pegloticase, asparaginase, taliglucerase alfa, plasmin, carboxypeptidase g2, glucarpidase, coagulation Factor XIII A, closulfase alfa, coagulation Factor X, asfotase alfa, sebelipase alfa, cerliponase alfta, vestronidase alfa-vjbk, pegvaliase-pqpz, and combinations thereof, or a polynucleotide encoding the same. In some embodiments, the present disclosure provides a population of pulmonary cells comprising a plurality of alveolar cells harboring at least one preselected enzyme known to enzymatically breakdown at least one toxin. In some embodiments, a population of pulmonary cells comprises a plurality of alveolar cells harboring an exogenously introduced enzyme selected from the group consisting of phenylalanine ammonia lyase (PAL), phenylalanine hydroxylase (PAH), galactose degrading enzyme, galactose-1-phosphate uridylyltransferase (GALT), 2-oxoisovalerate dehydrogenase subunit alpha, mitochondrial (BCKDHA), 2-oxoisovalerate dehydrogenase subunit beta, mitochondrial (BCKDHB), BDT complex enzymes, kynureninase, cocaine esterase, arginase, glutamine synthase, arginosuccinate lyase, arginosuccinate synthase, carbamyl phosphate synthetase I, N-acetylglutamate synthetase, ornithine transcarbamylase, ornithine translocase, nicotine oxidoreductase, uricase, acid alpha-glucosidase (GAA), thiosulfate sulfurtransferase (rhodanese), collagenase, asparaginase, anti-inhibitor coagulant complex, tissue-type plasminogen activator (t-Pa), Alteplase, pegademase bovine, alglucerase, imiglucerase, Factor IX, dnase, pancrelipase (amylase; lipase; protease), sacrosidase, truncated (non-glycosylated) t-PA (357 of 527aa), coagulation Factor VIIa, tissue plasminogen activator, antihemophilic factor (AHF), laronidase, agalsidase beta, hyaluronidase, galsulfase, idursulfase, alglucosidase alfa, thrombin, velaglucerase alfa, pegloticase, asparaginase, taliglucerase alfa, plasmin, carboxypeptidase g2, glucarpidase, coagulation Factor XIII A, elosulfase alfa, coagulation Factor X, asfotase alfa, sebelipase alfa, cerliponase alfta, vestronidase alfa-vjbk, pegvaliase-pqpz, and combinations thereof, or a polynucleotide encoding the same.

### Subject Populations

In some embodiments, a subject of the present disclosure (e.g., who undergoes methods of alleviating one or more negative effects of a toxin as described herein) can be, but is not limited to, a human or a non-human vertebrate. In some embodiments, a subject in accordance with the methods of the disclosure is a mammal. Mammals include, for example and without limitation, a household pet (e.g., a dog, a cat, a rabbit, a ferret, a hamster, etc.), a livestock or farm animal (e.g., a cow, a pig, a sheep, a goat, a pig, a chicken or another poultry), a horse (e.g., a thoroughbred horse), a monkey, a laboratory animal (e.g., a mouse, a rat, a rabbit, etc.), and the like. In a preferred embodiment, a subject of the present disclosure is a human. Technologies of the present disclosure can be practiced in any subject in need of treatment that is amenable to pulmonary delivery of an exogenously applied enzyme.

In some embodiments, methods of the present disclosure can be practiced in a subject that has a genetic condition, such as an enzyme deficiency, leading to accumulation of an endogenous metabolite. Non-limiting examples of genetic conditions involving an enzyme deficiency include phenylketonuria, hyperuricemia, gout, cystinuria, ornithine transcarbamylase deficiency (OTCD), galactosemia, maple syrup urine disease, and urea cycle disorder. In some embodiments, methods of the present disclosure can be practiced in a subject suffering from a condition involving secondary accumulation of an endogenous metabolite. In some embodiments, methods of the present disclosure can be practiced in a subject suffering from an autoimmune disease. In some embodiments, methods of the present disclosure can be practiced in a subject suffering from a cancer, such as leukemia. In some embodiments, methods of the present disclosure can be practiced in a subject suffering from a substance use disorder. Non-limiting examples of substance use disorders include cocaine use disorder, tobacco use disorder, nicotine use disorder, stimulant use disorder, sedative use disorder, and opioid use disorder.

In some embodiments, methods of the present disclosure can be practiced in a subject that has consumed or otherwise come into contact with a toxin, and said toxin has entered the subject's systemic circulation. In some embodiments, methods of the present disclosure can be practiced in a subject that has been poisoned. In some embodiments, methods of the present disclosure can be practiced in a subject contemplating a consumption of, is in a process of consuming, or has consumed one or more solid or liquid preparations comprising a toxin, such as a drug or other toxic agent. For example, in some embodiments, methods of the present disclosure can be practiced in a subject that has consumed or otherwise come into contact with cocaine, and said cocaine has entered the subject's systemic circulation. In some embodiments, methods of the present disclosure can be practiced in a subject that has consumed or otherwise come into contact with cyanide, and said cyanide has entered the subject's systemic circulation.

In some embodiments, a subject of the present disclosure is a human newborn (e.g., birth to 1 month of age), a human infant (e.g., 1 month to 1 year of age), a human child (1 year to 12 years of age), a human adolescent (13 years to 17 years of age), or a human adult (18 years of age or older).

### Combination Therapy

In some aspects, methods of the present disclosure further comprise administering of an additional therapeutic agent(s) and/or method to the subject (e.g., a “combination therapy”). Additional therapeutic agents may be administered to a subject by any route of introducing or delivering to a subject an additional therapeutic agent to perform its intended function. In some embodiments, a composition comprising at least one enzyme known to enzymatically breakdown at least one toxin and an additional therapeutic agent are administered by the same route of administration. In some embodiments, a composition comprising at least one enzyme known to enzymatically breakdown at least one toxin and an additional therapeutic agent are administered by the different routes of administration. For example, in some embodiments, a composition comprising at least one enzyme known to enzymatically breakdown at least one toxin is administered via inhalable or intratracheal administration, and an additional therapeutic agent is administered by oral or intravenous administration.

In some aspects, methods of the present disclosure further comprise administration of an additional therapeutic agent(s) and/or method(s) that can reduce the level of toxin in the systemic circulation of a subject (e.g., in the blood of a subject) and/or reduce adverse effects of toxin consumption, such as, for example, nausea, vomiting, confusion, seizures, and slowed breathing. In some embodiments, methods of the present disclosure are administered to a subject in combination with one or more of stomach pumping, intravenous saline drip, anti-nausea agents, and analgesic drugs.

In some embodiments, methods of the present disclosure further comprise administration of an additional therapeutic agent(s) and/or method(s) useful for the treatment of auto-brewery syndrome. Such additional therapeutic agent(s) and/or method(s) can include, for example, antifungals, antibiotics, probiotics, a low-carbohydrate diet. In some embodiments, methods of the present disclosure do not further comprise administration of antibiotics.

### EXAMPLES

These examples are provided for illustrative purposes only and not to limit the scope of the claims provided herein.

### Example 1. In Vivo Reduction of Circulating Phenylalanine Levels by PAL Deposition in Rat and Mouse Lung

The present Example describes the effect of phenylalanine ammonia lyase (PAL) enzyme deposition in the lung tissue of a mouse or rat on circulating Phenylalanine (Phe) levels.

To establish a chemically-induced model of a PKU disease state, mice and rats were administered p-chloro phenylalanine methyl ester (pCP), known in vivo inhibitor of phenylalanine hydroxylase (PAH), together with Phe, to simulate persistent elevated circulating Phe levels.

Each experimental group containing 4 rats were injected via the intraperitoneal (IP) injection with one dose of pCP-Phe (300 mg/kg+100 mg/kg) for 3 consecutive days. Blood was drawn on the morning of day 1 before pCP-Phe injection to determine the basal level of Phe in the blood. Additional blood draws were performed on day 3 and day 4. On day 4, an Anabaena variabilis phenylalanine ammonia lyase (PAL) enzyme was either injected via intravenous (IV) administration (35 mg/kg animal weight) or via intratracheal (IT) administration (55 mg/kg animal weight). Blood samples were then collected at 1, 2, 3, 6, and 19 hours after PAL administration. Blood samples were analyzed using the Abcam Phe detection kit.

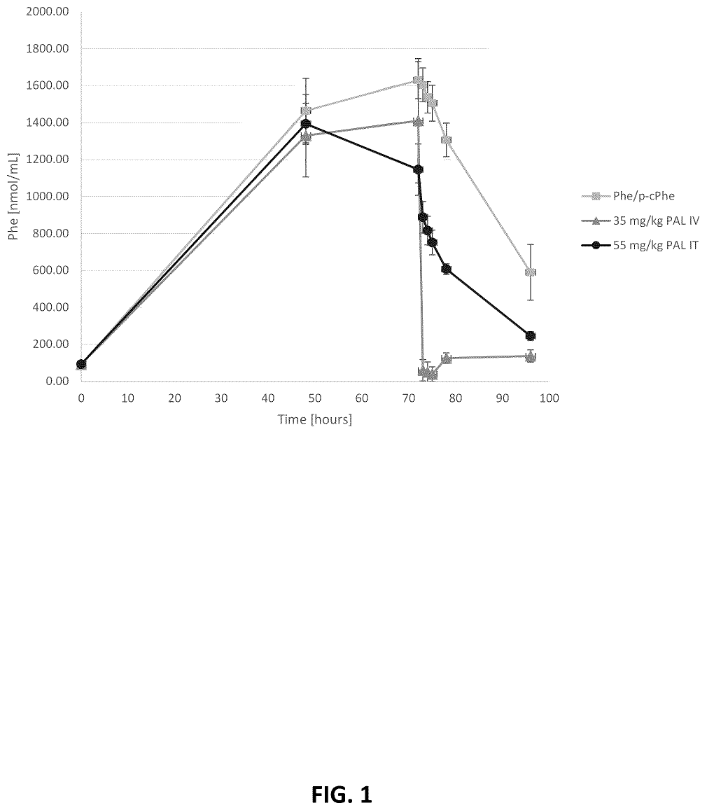

As shown in FIG. 1, accumulation of Phe in the 3 groups was observed, due to the inhibition of the rat native PAH by pCP and the additional delivery of Phe. On day 4, a gradual reduction in serum Phe levels was observed in the non-PAL treated group as expected, since PAH inhibition waned over time. However, the observed reduction in observed circulating Phe levels was significantly more robust in the PAL-treated animals. Surprisingly, IT administration of PAL yielded a reduction in Phe similar in magnitude to that observed following IV administration of PAL. Thus suggested that PAL deposited in the lung tissue could facilitate the breakdown of Phe even though the PAL was not delivered to the systemic circulation as achieved by the IV route.

A similar set of experiments were performed using a mouse model of PKU. Mice were treated with pCP/Phe to increase the level of Phe circulating in the blood, and were then administered PAL via subcutaneous (SQ) administration or IT administration. Briefly, 3 groups of mice (4 animals per group) were treated with a regimen of pCP/Phe delivered by IP administration for 2 days to increase the level of Phe in the blood. On day 3 following pCP/Phe delivery, one group received pCP alone for another 2 days, one group received pCP plus PAL (SQ), and one group received pCP plus PAL (IT). On day 4, blood was drawn from the animals to measure circulating Phe levels. A fourth group with 2 mice did not receive pCP nor PAL (untreated control group). Phe level from the collected blood samples were measured by LC-MS/MS analysis.

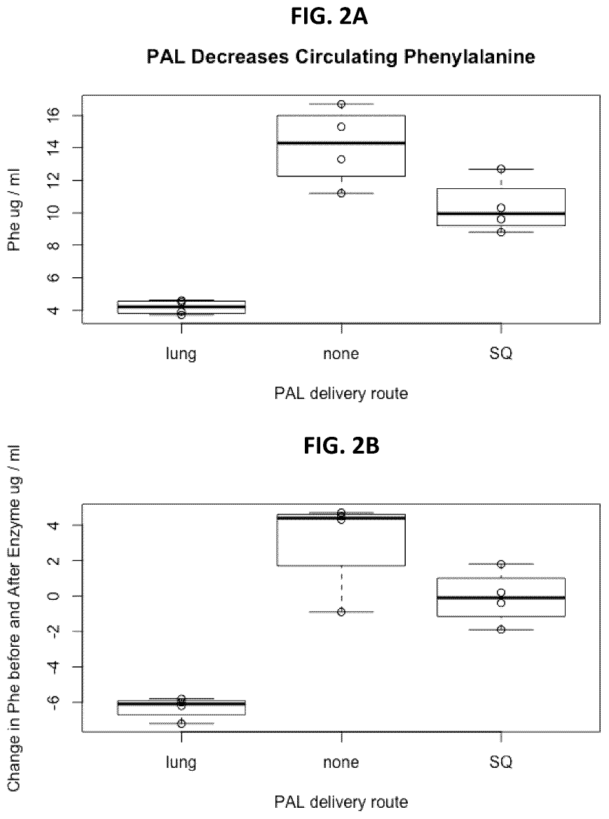

As shown in FIG. 2A and FIG. 2B, circulating phenylalanine levels were reduced in animals treated with pulmonary delivered PAL relative to animals that were not administered PAL, and relative to animals who were administered PAL via subcutaneous administration. These results suggest that the pulmonary deposition of a PAL enzyme is sufficient to reduce phenylalanine levels in a subject, thus protecting the subject from the physiological consequences of elevated phenylalanine levels. Such a treatment strategy may be applicable to subjects suffering from diseases resulting from diseases involving PAL deficiency and/or elevated blood phenylalanine levels, including phenylketonuria.

In a further set of experiments, a genetic mouse model of PAH deficiency was used to probe the efficacy of IT-administered PAL in reducing circulating Phe levels. Briefly, homozygous enu2/enu2 mice were bred from heterozygous mice obtained from the Jackson Laboratory (jax.org). Homozygous enu2/enu2 mice accumulate PHE due to a defective PAH gene.

Mice were administered PAL via IT or SQ administration preceding days 2, 3, and 4 of the week-long experiment. Blood samples were obtained from the mice each day of the 7-day time course, and were subjected to LC-MS/MS analysis.

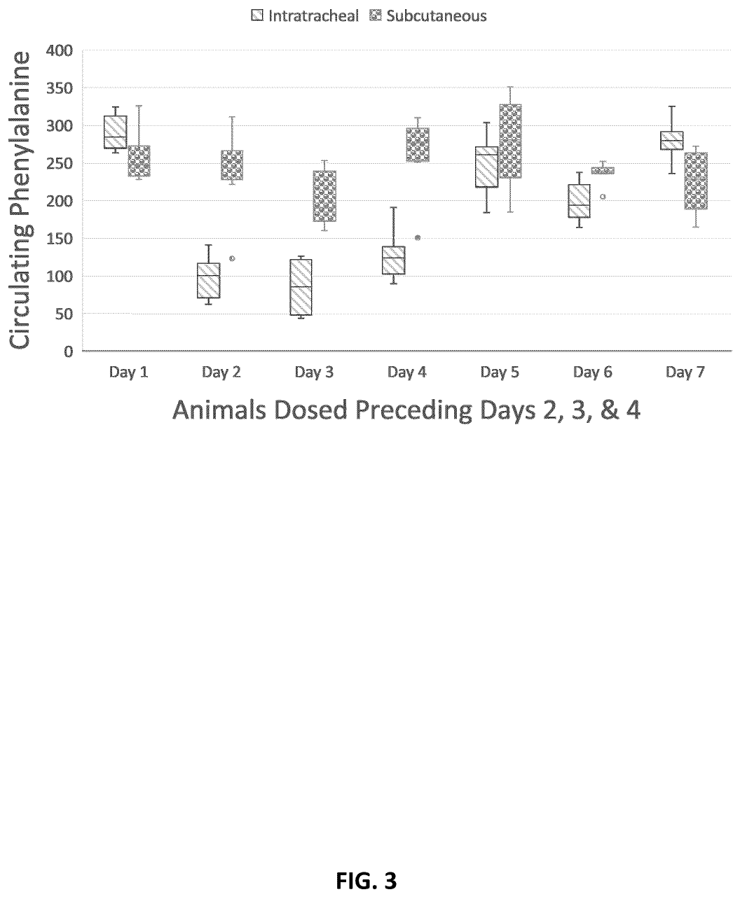

As shown in FIG. 3, PAL administration by IV and IT reduced circulating Phe levels in the PKU model mice. Plasma phenylalanine levels were reduced in PKU mice treated twice daily with PAL with both IT and SQ administration. Similar to what was seen in a chemically induced mouse model of PKU, efficacy of IT-delivered PAL was significantly higher than that observed of PAL delivered via SQ injection. On each day following treatment, IT-delivered PAL was significantly reduced compared to SQ PAL (FIG. 3; 51-59% reduction, p<0.001). By Day 5, after 1 day of washout, Phe levels in both groups had returned to pre-treatment (Day 1) baseline. Surprisingly, IT administration of PAL led to a more robust reduction of circulating PAL levels than that observed following SQ administration. This corroborated previous data suggesting that PAL deposited in the lung tissue could facilitate the breakdown of Phe even though the PAL was not delivered to the systemic circulation as achieved by the SQ route.

### Example 2. In Vivo Reduction of Circulating Uric Acid Levels by Uricase Deposition in Mouse Lung

The present Example describes the effect of uricase enzyme deposition in the lung tissue of a mouse on circulating uric acid levels.

Gout is a form of inflammatory arthritis characterized by recurrent attacks of a red, tender, hot, and swollen joint, caused by the deposition of needle-like crystals of uric acid known as monosodium urate crystals. Gout is a condition that results from persistently elevated levels of uric acid (urate) in the blood, termed hyperuricemia.

The enzyme urate oxidase (UO), uricase, or factor-independent urate hydroxylase, absent in humans, catalyzes the oxidation of uric acid to 5-hydroxyisourate. Thus, it was hypothesized that deposition of a uricase enzyme in the lung tissue of a mouse could reduce circulating uric acid levels.

Briefly, mice were 5 times administered a uricase enzyme suspended in a uricase stock solution, by either intratracheal (IT) administration or intravenous (IV) administration. The uricase stock solution was composed of Recombinant Aspergillus flavus uricase from E. coli (prospecbio.com/urate_oxidase). 20 mg of enzyme was suspended in 0.45 ml 50 mM borate buffer containing 0.001% Triton X-100 and 1.0 mM EDTA, at pH 8.5. 40 μL of stock solution was administered to each mouse, resulting in each mouse receiving approximately 2 mg of enzyme. Control mice were administered saline and no enzyme.

Following uricase enzyme administration, blood samples were obtained from the mice 30, 60, 120, 180, and 240 minutes after delivery. Blood samples were also obtained immediately before uricase enzyme delivery (time 0). Blood samples were analyzed using a Uric Acid Assay Kit (abcam.com/products/assay-kits/uric-acid-assay-kit-ab65344.html), for quantification of blood uric acid levels.

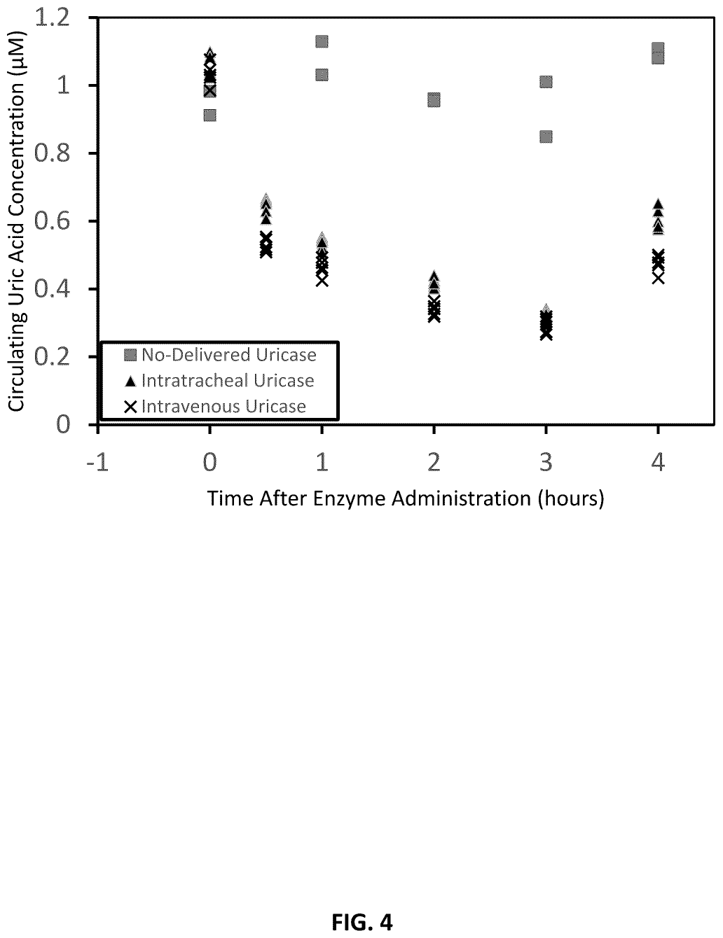

As shown in FIG. 4, administration of a uricase enzyme via IT or IV administration was sufficient to significantly reduce circulating uric acid levels at all tested time points. Surprisingly, the magnitude of the reduction in circulating uric acid levels achieved by IT administration was comparable to that achieved by IV administration, despite the fact that IT administration, unlike IV administration, does not result in systemic delivery of the uricase enzyme. Rather, IT administration results in deposition of the uricase enzyme in the lung tissue, where it is understood by the present inventors to act on uric acid diffusing out of the pulmonary blood supply and into the lung tissue. These data show that the lung can be transformed into a tunable metabolic organ capable of metabolizing a systemically circulating toxin to a magnitude sufficient to detect significant reductions in circulating levels of a systemic toxin. These data also suggest that pulmonary delivery of a uricase enzyme may be applicable to the treatment of diseases involving the systemic build-up of uric acid, such as hyperuricemia.

In a second experiment, mice (5 per group) were administered A. flavus uricase enzyme resuspended in sterile water by either intratracheal (IT) administration or intravenous (IV) administration. The prepared stock solution was 40 mg/mL. 50 μL of stock solution was administered to each mouse, resulting in each mouse receiving approximately 2 mg of enzyme. The control group (n=2) were administered saline (IT) with no enzyme. The mice were maintained on a commercial diet (LabDiet 5K52) and water purified by reverse osmosis, which was available ad libitum. The uricase was a commercial lyophilized A. flavus uricase (1 mg/vial, Prospec Bio) and was stored at −20° C. until the scheduled experimental day. The mice were 8 week-old female C57BL/6 mice obtained from Charles River Labs.

Following uricase enzyme administration, blood samples were obtained from the mice 30, 60, 120, 180, and 240 minutes after delivery. Blood samples were also obtained immediately before uricase enzyme delivery (time 0). Blood samples were analyzed by fluorometric or colorimetric assay, using a Uric Acid Assay Kit (AbCam #ab65344) for quantification of blood uric acid levels.

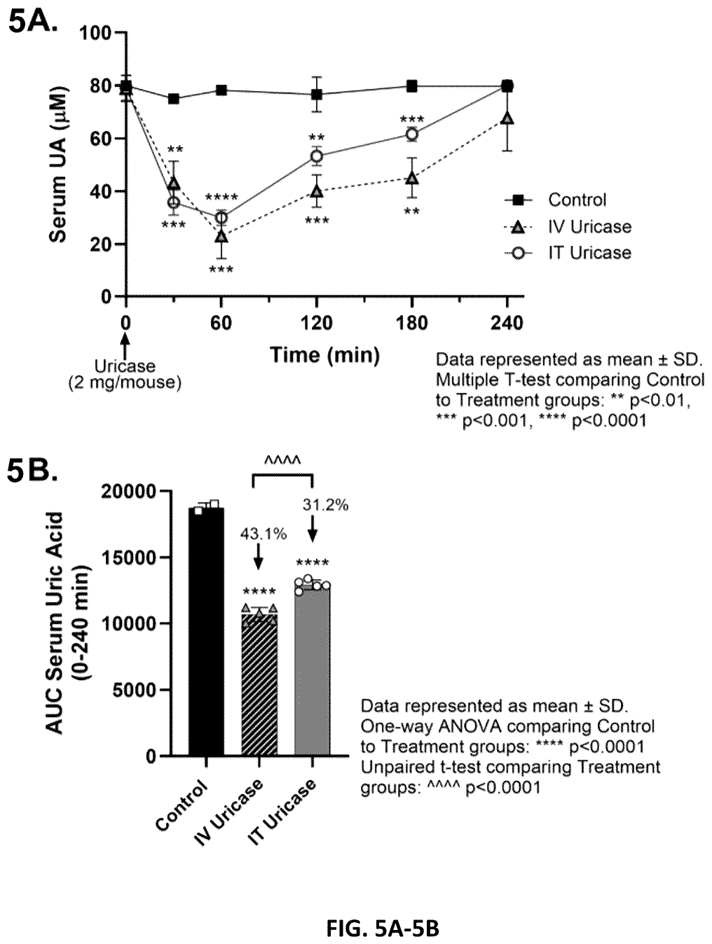

Circulating uric acid levels were significantly reduced in animals treated with 2 mg of a commercially available A. flavus uricase (FIG. 5A) independent of delivery route (IT vs IV). IT delivery decreased UA by 31.2% while IV delivery decreased it by 43.1% compared to untreated controls (AUC, FIG. 5B), which was a statistically significant improvement over IT (p<0.0001).

It was also noted that the mice receiving uricase via IV were more sluggish post-dose, while the IT-treated animals seemed to be fit with no observed changes in behavior.

These data further validate the potential of lung delivery of therapeutic enzymes, as well as indicate IT-delivery may have a better safety profile compared to systemic delivery. Taken together, this suggests lung-delivered therapeutics may have broad opportunities towards treatment of gout and other metabolic diseases.

### Example 3. In Vivo Reduction of Circulating Uric Acid Levels by Variant Uricase Deposition in Mouse and Monkey Lung

The present Example describes the effect of a variant uricase enzyme deposition in the lung tissue of mice and monkeys on circulating uric acid levels.

An induced model of hyperuricemia was produced in Cynomolgus non-human primates (NHP) by IV injection of uric acid (UA; 8.15 mg/kg) to assess the activity of an inhaled engineered variant uricase on UA in blood serum.

Briefly, on the morning of the experiment, 12 male non-naïve NHPs, 4-5 kg, were randomized into four groups of three animals: 1) Neg. Control: no treatment, no UA; 2) UA Control: no treatment, +UA control; 3) Low dose uricase+UA; 4) High dose uricase+UA. Animals had been fasted overnight. A baseline blood collection was obtained at time 0, and inhalation treatment was then administered via the Aeroneb® Solo vibrating mesh nebulizer system with spacer and oronasal mask within 15 minutes of initial blood draw. Nebulized treatment was administered for 20 minutes (uricase buffer, 60 mg/mL uricase, or 20 mg/mL uricase) with an inhaled target uricase dose of 25 mg/kg and 8.3 mg/kg in treatment groups. At 75 minutes, an IV injection of uric acid was provided (to induce hyperuricemia) or 0.9% saline (Control).

Blood samples were collected at 60- and 65-minutes (post-inhalation and pre-UA), and at 95-, 155-, 275-, and 515-minutes (post-inhalation and UA injection) corresponding to 1-, 2-, 4-, and 8-hours following completion of nebulized treatment. Food was provided to the animals after the final blood collection. A crossover design was used to maximize the number of NHPs per group (n=6 total), which incorporated a 7-day washout between each dosing day. Serum was isolated from blood samples using standard protocols and frozen at −80° C. until analysis by tandem mass spectrometry to quantify uric acid.

Healthy baseline UA, prior to inhalation treatment and UA injection, was 4 μM±3.4 SD, and Group 1 (Neg. Control) maintained this concentration throughout the time course. UA injection resulted in high UA in the serum (Cₘₐₓ=75 minutes) and exhibited a subsequent gradual decrease returning to baseline by ˜275 minutes for uricase-treated animals, while UA Control uric acid was still elevated at the end of the experiment.

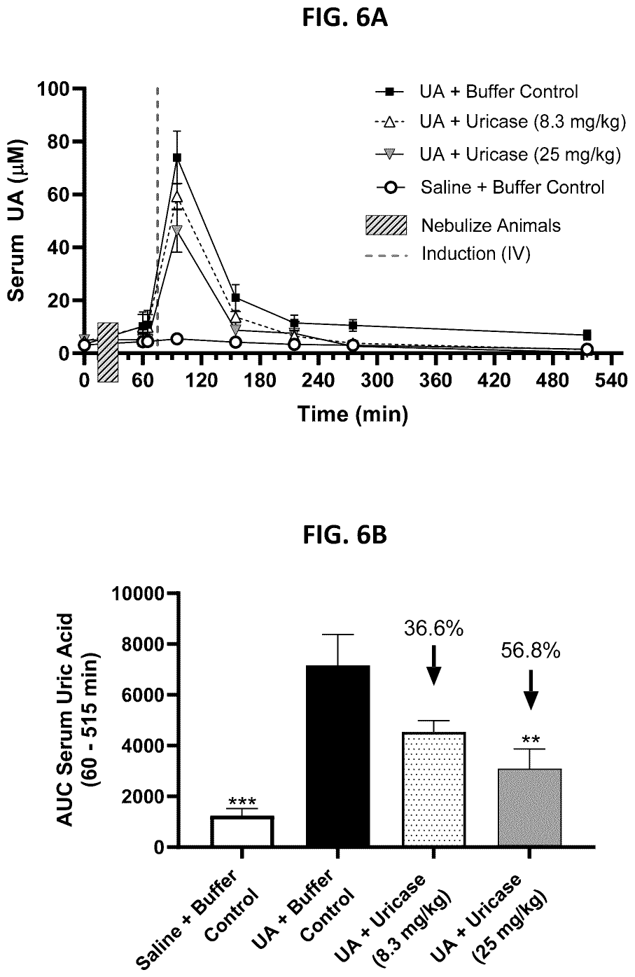

Variant uricase treatment targeted to the lungs resulted in a dose responsive reduction in circulating uric acid observable at all time points. For example, at Cₘₐₓ, UA serum concentration was 74 μM±24.5 SD (UA Control), 59 μM±12 SD (Low-dose uricase+UA), and 46 μM±20 SD (High-dose uricase+UA) (FIG. 6A). Serum uric acid was decreased 36.6% (Low-dose uricase) and 56.8% (High-dose uricase, p<0.01) compared to UA Control, as determined by area under the curve analysis of time course data (60-515 minutes). (FIG. 6B). No adverse effects of any kind were observed throughout the study.

In a second experiment, the ability of the variant uricase to decrease uric acid levels in vivo following lung deposition in vivo was assessed by directly comparing its activity to that of a WT uricase in mice. Briefly, mice (3 per group) were administered either 2 mg/mouse of uricase (A. flavus WT uricase enzyme or engineered A. globiformis variant uricase) or vehicle (uricase buffer) by intratracheal (IT) administration. The mice were maintained on a commercial diet (LabDiet 5K52) and water purified by reverse osmosis, which was available ad libitum. Uricase was prepared on the day of dosing. Animals received 2 mg/mouse in a 50 μL dosing volume, by direct deposition into the lungs (IT).

Following uricase enzyme administration, blood samples were obtained from the mice 30, 60, 120, 180, and 240 minutes after delivery. Blood samples were also obtained immediately before uricase enzyme delivery (time 0). Blood samples were analyzed by fluorometric or colorimetric assay using a Uric Acid Assay Kit (AbCam #ab65344) for quantification of blood uric acid levels.

A head-to-head comparison of the commercial A. flavus WT uricase and an engineered A. globiformis variant uricase was performed, in which 2 mg of enzyme was delivered to the lungs of healthy female mice. Serum uric acid levels were then measured to determine efficacy compared to untreated controls.

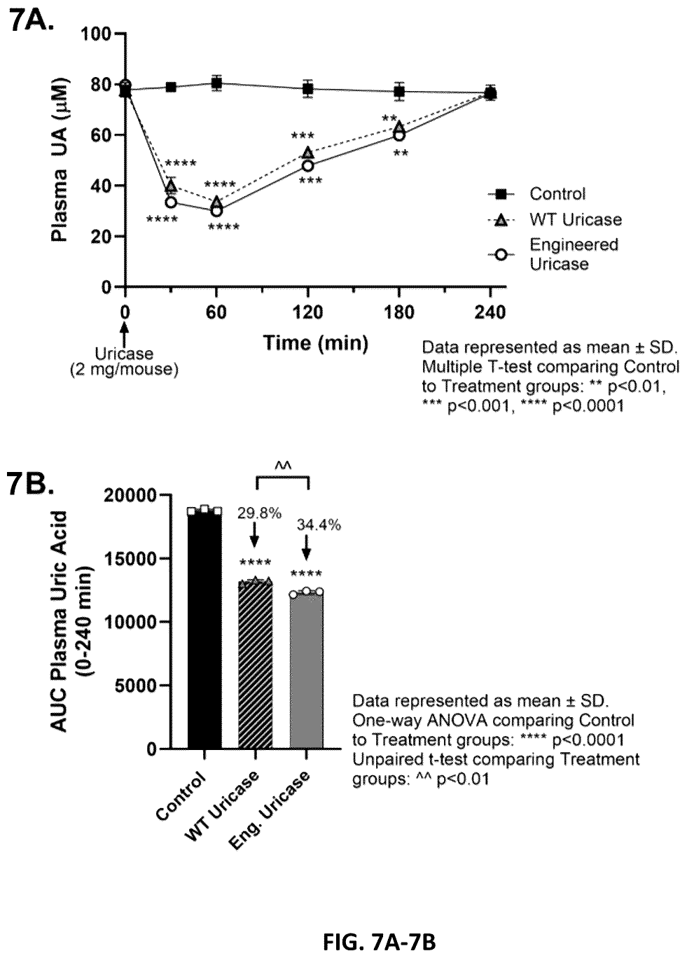

Circulating uric acid levels were significantly reduced in all treated animals (FIG. 7). The observed decrease in UA levels was similar independent of the enzyme in the time course analysis (FIG. 7A). WT delivery decreased UA by 29.8% while variant uricase delivery decreased it by 34.4% compared to untreated controls (AUC), which was a statistically significant improvement over WT (p<0.01) (FIG. 7B).

These data further validate the potential benefits of lung delivery of therapeutic enzymes, which can be enhanced using protein engineering techniques to improve enzyme stability in the lung, which may have broad opportunities towards treatment of gout and other metabolic diseases.

### Example 4. In Vivo Reduction of Circulating Cyanide Levels by Thiosulfate Sulfurtransferase (Rhodanese) Deposition in Mouse Lung

The present Example describes the effect of rhodanese enzyme deposition in the lung tissue of a mouse on circulating cyanide levels in a mouse model of cyanide poisoning.

Briefly, mice are administered potassium cyanide (KCN) in Na₂CO₃ via intraperitoneal (IP) injection, to establish an animal model of cyanide poisoning. Possible administration amounts include 200 μL of 10 mM or 20 mM KCN. The effect of rhodanese+sodium thiosulfate on cyanide-related lethality is assessed by measuring mouse death rates related to KCN administration, and in particular, changes in the KCN LD50. Rhodanese+sodium thiosulfate can be administered via intratracheal (IT) administration before or after KCN IP administration such as greater than 5 minutes before or greater than 5 minutes after KCN IP administration. In some experiments, rhodanese is administered via IT without sodium thiosulfate. Control conditions include a negative control in which no KCN is administered, and a positive control in which KCN is administered IP alone. In another experimental condition, of rhodanese+sodium thiosulfate can be administered intravenously (IV) to compare the efficacy relative to that observed following IT delivery.

The rhodanese enzyme can be a commercially available rhodanese enzyme (e.g., sigmaaldrich.com/US/en/product/sigma/r1756). Sodium thiosulfate can also be obtained commercially (e.g., sigmaaldrich.com/US/en/product/sial/phr2690).

It is expected that IT administration of rhodanese+sodium thiosulfate will promote survival of KCN-administered mice, and will increased the KCN LD50 of such mice.

### Example 5. In Vivo Reduction of Circulating Ammonia Levels by Glutamine Synthase Deposition in Mouse Lung

The present Example describes the effect of glutamine synthase enzyme deposition in the lung tissue of a mouse on circulating ammonia levels.

Hyperammonemia is a metabolic condition characterized by the raised levels of ammonia, a nitrogen-containing compound. Normal levels of ammonia in the body vary according to age. Hyperammonemia can result from various congenital and acquired conditions in which it may be the principal toxin.

Glutamine synthetase (GS) (EC 6.3.1.2) is an enzyme that plays an essential role in the metabolism of nitrogen by catalyzing the condensation of glutamate and ammonia to form glutamine: Glutamate+ATP+NH3→Glutamine+ADP+phosphate

Glutamine synthetase uses ammonia produced by nitrate reduction, amino acid degradation, and photorespiration. The amide group of glutamate is a nitrogen source for the synthesis of glutamine pathway metabolites.

The enzyme used in the following experiment was glutamine synthase, also known as glutamate-ammonia ligase.

In a first set of experiments, Applicant performed experiments in an induced hyperammonemia mouse model in which wildtype mice were injected with ammonium chloride (NH₄Cl). Briefly, ammonia levels were increased in these mice by injecting NH₄Cl, and glutamate and NH₄Cl levels were analyzed following glutamine synthase (GS) deposition in the lungs. Mice were administered saline, GS, or GS-glutamate by IT administration 10 minutes before delivery of NH₄Cl by intraperitoneal (IP) administration. The glutamine synthase stock solution was composed of L glutamine synthase from E. coli (sigmaaldrich.com/US/en/product/sigma/g1270). 300 μg of GS enzyme was suspended in 300 μL of water, resulting in a stock solution of 1 U/uL. For the control group, 40 μL of saline was administered to each mouse. For the GS IT NH₄Cl group, 20 μL of stock solution plus 20 μL of water was administered to each mouse, resulting in each mouse receiving approximately 20 units of enzyme. For the GS-glutamate IT NH₄Cl group, 20 μL of stock solution plus 20 μL of glutamate stock solution was administered to each mouse, resulting in each mouse receiving approximately 20 units of enzyme and 11.8 ug of glutamate.

10 minutes following glutamine synthase enzyme administration, NH₄Cl was injected by IP administration at 7 mg/kg. Blood samples were obtained from the mice 5, 15, 30, 45, 60, 80, and 120 minutes after injection. Blood samples were also obtained immediately before NH₄Cl delivery (time 0). Blood samples were analyzed using an ammonia detection kit (sigmaaldrich.com/US/en/product/sigma/mak538), for quantification of blood ammonia levels, a glutamine detection kit (abcam.com/en-us/products/assay-kits/glutamine-assay-kit-colorimetric-ab197011) for quantification of blood glutamine levels, and a glutamate detection kit (abcam.com/en-us/products/assay-kits/glutamate-assay-kit-ab83389) for quantification of blood glutamate levels.

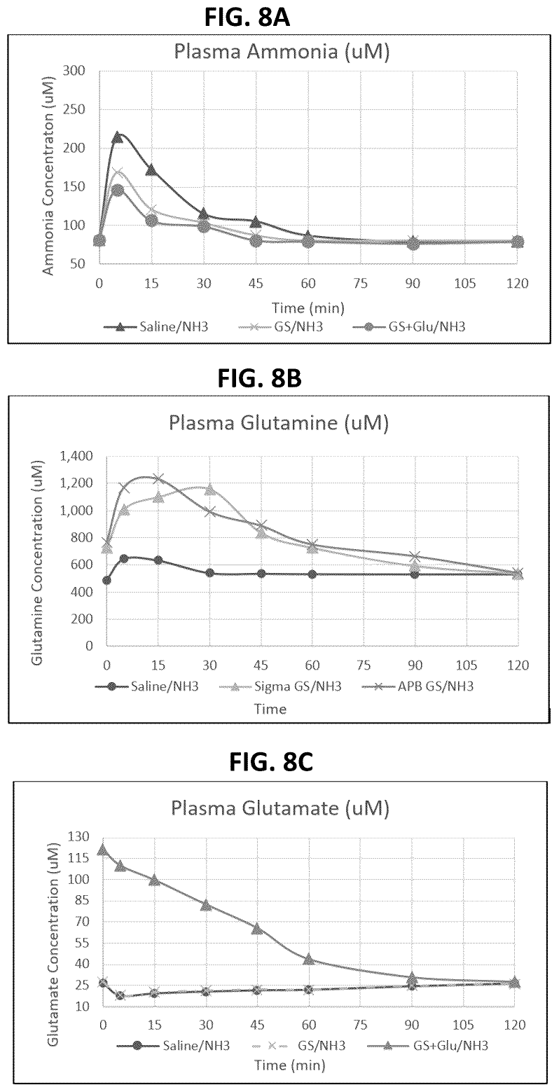

As shown in FIG. 8A, administration of a GS enzyme via IT administration was sufficient to significantly reduce circulating ammonia levels. Such a reduction in circulating plasma ammonia levels relative to the control group was enhanced by supplementing the reaction with glutamate. As shown in FIG. 8B and FIG. 8C, glutamine levels were increased in the presence of GS and GS+glutamate, and glutamate levels decreased over time, each of which is consistent with the biochemical activity of glutamine synthase (i.e., glutamate-ammonia ligase), respectively.

In a second set of experiments, Applicant assessed the ability of IT-administered GS enzyme to reduce circulating ammonia levels in an Ornithine transcarbamylase deficient (OTCD) mouse model.

Ornithine transcarbamylase deficiency (OTCD) is an X-linked liver disorder caused by partial or total loss of OTC enzyme activity. It is characterized by elevated plasma ammonia.

Briefly, OTCD mice were obtained from Jackson Labs (strain #001811, jax.org/strain/001811). A WT mouse control group was also included. Mice were administered saline or GS by IT administration 10 minutes before delivery of NH4Cl by intraperitoneal (IP) administration. The glutamine synthase stock solution was composed of L glutamine synthase (GS) from E. coli, the GS having the same sequence as the GS of sigmaaldrich.com/US/en/product/sigma/g1270. GS enzyme was suspended in water and diluted to produce a stock solution with a concentration of 1 μg/uL. For the WT control group and the OTC control group, 20 μL of saline was administered to each mouse. For the OTC GS-IT treated group, 20 μL of stock GS solution was administered to each mouse, resulting in each mouse receiving approximately 20 μg of enzyme.

10 minutes following glutamine synthase enzyme administration, NH₄Cl was injected by IP administration at 7 mg/kg. Blood samples were obtained from the mice 5, 15, 30, 45, 60, 80, and 120 minutes after injection. Blood samples were also obtained immediately before NH₄Cl delivery (time 0). Blood samples were analyzed using an ammonia detection kit (sigmaaldrich.com/US/en/product/sigma/mak538), for quantification of blood ammonia levels, a glutamine detection kit (abcam.com/en-us/products/assay-kits/glutamine-assay-kit-colorimetric-ab197011) for quantification of blood glutamine levels, and a glutamate detection kit (abcam.com/en-us/products/assay-kits/glutamate-assay-kit-ab83389) for quantification of blood glutamate levels.

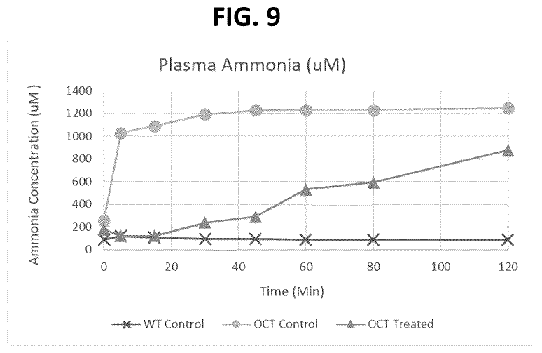

As shown in FIG. 9, administration of a GS enzyme via IT administration was sufficient to significantly reduce the accumulation of ammonia in the plasma of ammonia-challenged mice.

Taken together, these data suggest that pulmonary delivery of a glutamate synthase enzyme may be applicable to the treatment of diseases involving the systemic build-up of ammonia, such as hyperammonemia and Ornithine transcarbamylase deficiency (OTCD).

### Example 6. In Vivo Reduction of Circulating Arginine Levels by Arginase Deposition in Mouse Lung

The present Example describes the effect of arginase enzyme deposition in the lung tissue of a mouse on circulating arginine levels.

Arginase deficiency is an inherited disorder that causes the amino acid arginine (hyperargininemia) and ammonia to accumulate gradually in the blood. Ammonia, which is formed when proteins are broken down in the body, is toxic if levels become too high. The nervous system is especially sensitive to the effects of excess ammonia.

Arginase deficiency usually becomes evident by about the age of 3. It most often appears as stiffness, especially in the legs, caused by abnormal tensing of the muscles (spasticity). Other symptoms may include slower than normal growth, developmental delays and eventual loss of developmental milestones, intellectual disabilities, seizures, tremors, and difficulty with balance and coordination (ataxia). Occasionally, high-protein meals or stress caused by illness or periods without food (fasting) may cause ammonia to accumulate more quickly in the blood. This rapid increase in ammonia may lead to episodes of irritability, refusal to eat, and vomiting.

In some affected individuals, the signs and symptoms of arginase deficiency may be less severe and may not appear until later in life.

L-arginase hydrolyzes L-arginine into L-ornithine and urea and consequently leads to ammonia accumulation.

Briefly, mice were 4 times administered an arginase enzyme suspended in an arginase stock solution, by intratracheal (IT) administration. The arginase stock solution was composed of L-arginase from bovine liver (sigmaaldrich.com/US/en/product/sigma/a3233?utm_source=google&utm_medium=cpc&utm_campaign=8691857242&utm_content=98395646591&gbraid-0AAAAAD8KLQQ4EXPn2pu6CMF4WyzF4-npu&gclid=CjwKCAjwnK60BhA9EiwAmpHZw-wal-letyOPj_OY-AbkQpqEollpgOT4c6KsFjgF_le-M1MGCm8f1RoCSUQQAvD_BwE). 8 mg of enzyme was suspended in 105 μL of water. 25 μL of stock solution was administered to each mouse, resulting in each mouse receiving approximately 2 mg of enzyme. Control mice were administered saline and no enzyme.

Following arginase enzyme administration, blood samples were obtained from the mice 15, 30, 60, 120, 180, and 240 minutes after delivery. Blood samples were also obtained immediately before arginase enzyme delivery (time 0). Blood samples were analyzed using an L-arginine detection kit (abcam.com/en-us/products/assay-kits/l-arginine-assay-kit-ab241028), for quantification of blood L-arginine levels, and a urea detection kit (sigmaaldrich.com/US/en/product/sigma/mak471) for quantification of blood urea levels.

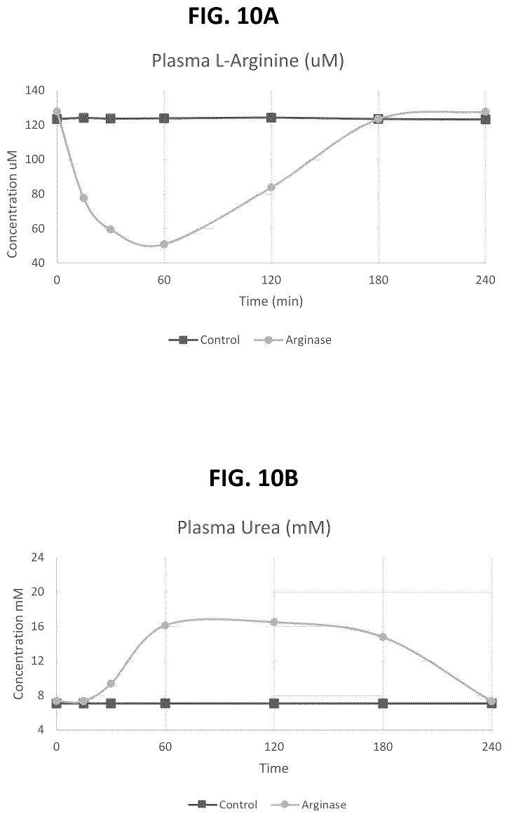

As shown in FIG. 10A, administration of an arginase enzyme via IT administration was sufficient to significantly reduce circulating L-arginine levels. L-arginine levels were reduced up to 3-hours post-administration of arginase enzyme. As shown in FIG. 10B, the mice treated with arginase accumulated plasma urea, a by-product of the degradation of arginine by the arginase enzyme. These data also suggest that pulmonary delivery of an arginase enzyme may be applicable to the treatment of diseases involving the systemic build-up of L-arginine, such as hyperargininemia and arginase deficiency.

### Example 7. In Vivo Reduction of Circulating Asparagine Levels by Asparaginase Deposition in Mouse Lung

The present Example describes the effect of asparaginase enzyme deposition in the lung tissue of a mouse on circulating asparagine levels.

Acute lymphoblastic leukemia (ALL) is a rare hematologic malignancy resulting in the production of abnormal lymphoid precursor cells. Occurring in B-cell and T-cell subtypes, ALL is more common in children, comprising nearly 30% of pediatric malignancies, but also constitutes 1% of adult cancer diagnoses. Outcomes are age-dependent, with five-year overall survival of greater than 90% in children and less than 20% in older adults. L-asparaginase depletes serum levels of L-asparagine. As leukemic cells are unable to synthesize this amino acid, its deprivation results in cell death. The success of asparaginase-containing regimens in the treatment of pediatric ALL, and poor outcomes with conventional cytotoxic regimens in adults, have led to trials of pediatric or pediatric-inspired regimens incorporating asparaginase in the adolescent and young adult and adult populations. See Juluri et al, Blood and Lymphatic Cancer: Targets and Therapy 2022.

Asparaginase converts asparagine to aspartic acid and releases ammonia.

Briefly, mice were 4 times administered an asparaginase enzyme suspended in an asparaginase stock solution, by intratracheal (IT) administration. The asparaginase stock solution was composed of asparaginase from E. Coli (sigmaaldrich.com/US/en/product/sigma/a3809). 2 mg of enzyme was suspended in 100 μL of water. 25 μL of stock solution was administered to each mouse, resulting in each mouse receiving approximately 100 units of enzyme (at 100-300 units/mg). Control mice were administered saline and no enzyme. Mice were fed a regular diet before and during the experiment.

Following asparaginase enzyme administration, blood samples were obtained from the mice 5, 15, 30, 60, 120, 180, and 240 minutes after delivery. Blood samples were also obtained immediately before asparaginase enzyme delivery (time 0). Blood samples were analyzed using an asparagine detection kit (abcam.com/en-us/products/assay-kits/asparagine-assay-kit-fluorometric-ab273333), for quantification of blood asparagine levels, an aspartate detection kit (abcam.com/en-us/products/assay-kits/aspartate-assay-kit-ab102512) for quantification of blood aspartate levels, and an ammonia detection kit (sigmaaldrich.com/US/en/product/sigma/mak538) for quantification of blood ammonia levels.

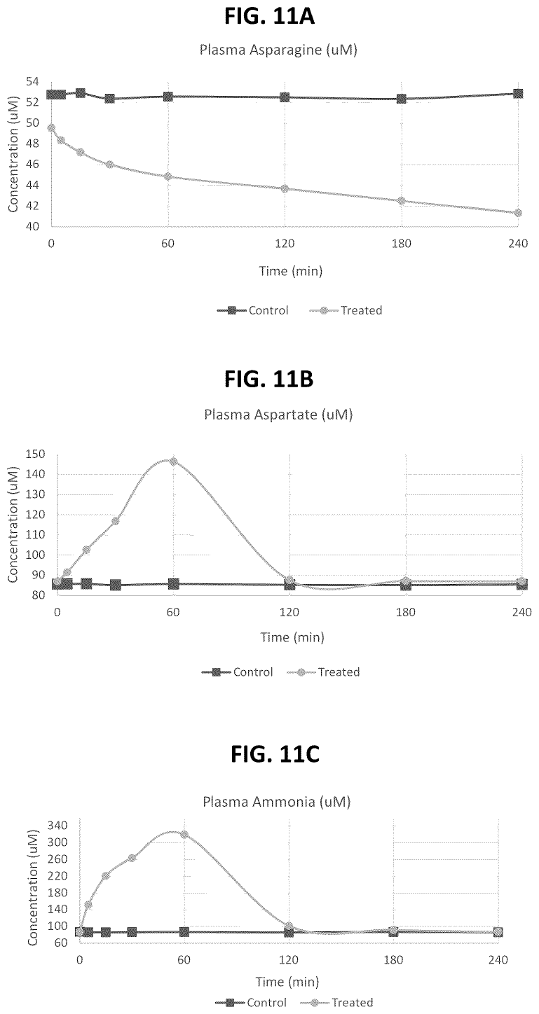

As shown in FIG. 11A, administration of an asparaginase enzyme via IT administration was sufficient to significantly reduce circulating asparagine levels. Asparagine levels were reduced 4-hours post-administration of asparaginase enzyme, which was the longest timepoint tested. The mice treated with asparaginase transiently accumulated plasma aspartate (FIG. 11B), and plasma ammonia (FIG. 11C), by-products of the degradation of asparagine by the asparaginase enzyme. These data also suggest that pulmonary delivery of an asparaginase enzyme may be applicable to the treatment of diseases for which reduction of circulating asparagine levels may be advantageous, such as ALL.

These examples are provided for illustrative purposes only and not to limit the scope of the claims provided herein.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, agents, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method of alleviating one or more negative effects of a toxin in vivo comprising:
(a) introducing at least one preselected enzyme to pulmonary tissue of a subject, said at least one preselected enzyme being known to enzymatically breakdown at least one toxin that is exerting one or more negative effects against said subject; and
(b) allowing the enzymatic breakdown of said at least one toxin to take place in vivo, alleviating said one or more negative effects.

2. The method of claim 1, in which said subject is afflicted with one or more genetic disorders.

3. The method of claim 2, in which said one or more genetic disorders is selected from the group consisting of phenylketonuria, gout, cystinuria, ornithine transcarbamylase deficiency (OTCD), galactosemia, maple syrup urine disease, tumor suppression disorder, cocaine use disorder, urea cycle disorder, tobacco use disorder, Pompe's disease, sucrase isomaltase deficiency, arginase deficiency, hyperargininemia, and combinations thereof.

4. The method of claim 1, in which said subject suffers from a condition that results in an accumulation of said at least one toxin.

5. The method of claim 1, in which said subject has been exposed to said at least one toxin.

6. The method of claim 1, in which said at least one toxin is selected from the group consisting of phenylalanine, uric acid, cystine, arginine, lysine, ornithine, leucine, isoleucine, valine, an amino acid, galactose, kynurenine, cocaine, ammonia, nicotine, cyanide, organophosphates, and combinations thereof.

7. The method of claim 1, in which said at least one preselected enzyme is selected from the group consisting of phenylalanine ammonia lyase (PAL), phenylalanine hydroxylase (PAH), galactose degrading enzyme, galactose-1-phosphate uridylyltransferase (GALT), 2-oxoisovalerate dehydrogenase subunit alpha, mitochondrial (BCKDHA), 2-oxoisovalerate dehydrogenase subunit beta, mitochondrial (BCKDHB), BDT complex enzymes, kynureninase, cocaine esterase, arginase, glutamine synthase, arginosuccinate lyase, arginosuccinate synthase, carbamyl phosphate synthetase I, N-acetylglutamate synthetase, ornithine transcarbamylase, ornithine translocase, nicotine oxidoreductase, uricase, acid alpha-glucosidase (GAA), thiosulfate sulfurtransferase (rhodanese), collagenase, asparaginase, anti-inhibitor coagulant complex, tissue-type plasminogen activator (tPa), Alteplase, pegademase bovine, alglucerase, imiglucerase, Factor IX, dnase, pancrelipase, sacrosidase, truncated (non-glycosylated) t-PA (357 of 527aa), coagulation Factor VIIa, tissue plasminogen activator, antihemophilic factor (AHF), laronidase, agalsidase beta, hyaluronidase, galsulfase, idursulfase, alglucosidase alfa, thrombin, velaglucerase alfa, pegloticase, taliglucerase alfa, plasmin, carboxypeptidase g2, glucarpidase, coagulation Factor XIII A, elosulfase alfa, coagulation Factor X, asfotase alfa, sebelipase alfa, cerliponase alfta, vestronidase alfa-vjbk, pegvaliase-pqpz, and combinations thereof.

8. The method of claim 1, in which said at least one toxin is an amino acid or an acid.

9. The method of claim 1, in which said pulmonary tissue exposes said at least one toxin by an action of the subject's circulatory system.

10. The method of claim 1, in which introducing comprises contacting pulmonary tissue with either a polypeptide having an amino acid sequence corresponding to said at least one preselected enzyme, a polynucleotide encoding said at least one polypeptide, or both.

11. The method of claim 1, in which introducing comprises transducing a plurality of cells present in pulmonary tissue to express said at least one preselected enzyme.

12. The method of claim 11, in which the plurality of cells is transduced with DNA, mRNA, or a construct thereof.

13-16. (canceled)

17. A method of transforming a lung of a subject to include at least one enzymatic breakdown function comprising introducing to a lung of a subject at least one preselected enzyme known to facilitate an enzymatic breakdown of at least one toxin that, if present, is present systemically in the subject.

18-21. (canceled)

22. A transformed pulmonary cell comprising an alveolar cell harboring at least one preselected enzyme known to enzymatically breakdown at least one toxin via an introduction of either a polypeptide having an amino acid sequence corresponding to said preselected enzyme, a polynucleotide encoding said polypeptide, or both.

23-25. (canceled)

26. A method of alleviating one or more negative effects of a condition in a subject, comprising:
(a) introducing asparaginase to pulmonary tissue of a subject diagnosed with leukemia, and
allowing asparaginase to enzymatically break down asparagine in vivo, thereby alleviating said one or more negative effects; or
(b) introducing oxalate decarboxylase to pulmonary tissue of a subject diagnosed with oxalosis, primary hyperoxaluria, or secondary hyperoxaluria, and
allowing the oxalate decarboxylase to enzymatically break down oxalate; or
(c) introducing phenylalanine ammonia lyase (PAL) to pulmonary tissue of a subject with phenylketonuria, and
allowing the PAL to enzymatically break down phenylalanine; or
(d) introducing uricase to said pulmonary tissue of a subject with hyperuricemia, and
allowing the uricase to enzymatically break down uric acid; or
(e) introducing thiosulfate sulfurtransferase said pulmonary tissue of a subject with cyanide poisoning, and
allowing the thiosulfate sulfurtransferase to enzymatically break down of said cyanide; or
(f) introducing glutamine synthase to pulmonary tissue of a subject with hyperammonemia, and
allowing the glutamine synthase to enzymatically break down ammonia; or
(g) introducing arginase to pulmonary tissue of a subject with hyperargininemia; and
allowing the arginase to enzymatically break down arginine.

27-39. (canceled)

40. The method of claim 1, in which said at least one preselected enzyme resides substantially in the subject's pulmonary tissue.

41. The method of claim 1, in which said at least one preselected enzyme is: (i) introduced under conditions that inhibit or do not support systemic delivery of said at least one preselected enzyme to said subject, (ii) introduced in a manner that does not include pulmonary transmucosal delivery of said at least one preselected enzyme to said subject; or (iii) introduced to the at least a portion of the lung of said subject in a manner that minimizes a systemic introduction into said subject.

42-43. (canceled)

44. The method of claim 1, in which said at least one preselected enzyme, a variant thereof, or a combination thereof excludes an ADH/KRED bound to at least one long-acting molecule or complexing molecule is excluded.

45. The method of claim 1, in which an introduction excludes an inhalation of an atomized solution of an ADH/KRED bound to at least one long-acting molecule or complexing molecule.

46-54. (canceled)
